(19) United States
(12) Patent Application Publication
(10) Pub. No.: US 2024/0076723 A1
(43) Pub. Date: Mar. 7, 2024

(54) CLEAVAGE OF CAPTURE PROBES FOR SPATIAL ANALYSIS

(71) Applicant: 10x Genomics, Inc., Pleasanton, CA (US)

(72) Inventor: Marco Mignardi, Sundbyberg (SE)

(21) Appl. No.: 18/269,634

(22) PCT Filed: Dec. 30, 2021
(86) PCT No.: PCT/US2021/065746

§ 371 Date: Jun. 26, 2023

Related U.S. Application Data
(60) Provisional application No. 63/132,103, filed on Dec. 30, 2020.

(51) Int. Cl. C12Q 1/6841; C12Q 1/6837

(52) U.S. Cl. CPC C12Q 1/6841; C12Q 1/6837; C12Q 2600/16

(57) ABSTRACT

Provided herein are methods of determining a location of an analyte in a biological sample, and systems for performing these methods. In particular, the methods and systems comprise decreasing the binding events of an analyte from a biological sample to capture probes that surround a biological sample positioned on a substrate.

### CROSS-REFERENCE TO RELATED APPLICATIONS

This application claims the benefit of U.S. Provisional Application Ser. No. 63/132,103, filed Dec. 30, 2020, the entire contents of which is incorporated by reference herein.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or provide significant analyte data from individual, single cells, but fails to provide information regarding the position of the single cells from the originating biological sample (e.g., tissue).

In some workflows, spatial heterogeneity can be studied by positioning a biological sample on a substrate. A substrate can include capture probes with capture domains which can bind to analytes in the biological sample. During an assay, sensitivity and specificity can decrease when analytes bind to capture probes outside of the biological sample (e.g., not covered by the biological sample). The binding of an analyte to a capture domain of a capture probes outside of the biological sample (e.g., not covered by the biological sample) can potentially result in a waste of resources such as unnecessary costs attributed to skewed analyses, decreased sensitivity of the assay, and unnecessary sequencing reads due to analytes captured outside of the biological sample.

### SUMMARY

In order to increase efficiency, sensitivity, and/or decrease the binding of analytes to capture domains of capture probes on an area of an array not covered by the biological sample, provided herein are methods of cleaving the capture probes that are located in an area of an array that is not covered by the biological sample. In some cases, capture probes can comprise a photocleavable linker. In some cases, the area of the array not covered by the biological sample can be contacted by light to cleave a photocleavable linker of one or more of the capture probes in the area of the array not covered by the biological sample.

In other embodiments, the capture probes can comprise a cleavage sequence and the area of the array not covered by the biological sample can be contacted with a cleavage probe. Subsequently, the area of the array not covered by the biological sample is contacted with an enzyme that cleaves the cleavage sequence hybridized to the cleavage probe, thereby releasing the capture probe from the area of the array not covered by the biological sample.

In one aspect, provided herein is a method for determining a location of an analyte in a biological sample, the method comprising (a) contacting a biological sample with an array comprising a plurality of capture probes, wherein a capture probe of the plurality of capture probes comprises (i) a photocleavable linker; (ii) a spatial barcode, and (iii) a capture domain; (b) exposing the area of the array not covered by the biological sample with a wavelength of light that cleaves the photocleavable linker of the capture probes, thereby releasing the capture probes from the area of the array not covered by the biological sample; (c) removing the cleaved capture probes; (d) contacting an analyte in the biological sample with a capture domain of a capture probe in an area of the array covered by the biological sample; and (e) determining (i) the sequence of the spatial barcode of the capture probe in the area of the array covered by the biological sample, or a complement thereof, and (ii) all or a portion of the sequence of the analyte bound to the capture domain of the capture probe in the area of the array covered by the biological sample, or a complement thereof, and using the sequences of (i) and (ii) to determine the location of the analyte in the biological sample.

In another aspect, provided herein are methods of decreasing hybridization of analytes in a biological sample to capture probes in an area of an array that is not covered by the biological sample that include: (a) contacting the biological sample with the array, where the array comprises a plurality of capture probes comprising (i) a photocleavable linker; (ii) a spatial barcode, and (iii) a capture domain; and (b) exposing the area of the array not covered by the biological sample with a wavelength of light that cleaves the photocleavable linker of the capture probes, thereby releasing the capture probes from the area of the array not covered by the biological sample, thereby decreasing hybridization of analytes in the biological sample to capture probes in the area of the array that is not covered by the biological sample.

In some embodiments, the method further includes, prior to step (b), imaging the biological sample on the array. In some embodiments, the imaging is performed using expansion microscopy, bright field microscopy, dark field microscopy, phase contrast microscopy, electron microscopy, fluorescence microscopy, reflection microscopy, interference microscopy, confocal microscopy, and visual identification, or any combination thereof. In some embodiments, the imaging is performed using phase contrast microscopy.

In some embodiments, methods described herein further comprise staining the biological sample. In some embodiments, the staining comprises the use of acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or any combination thereof. In some embodiments, the staining comprises the use of eosin and hematoxylin. In some embodiments, the staining comprises the use of radioisotopes, fluorophores, chemiluminescent compounds, bioluminescent compounds, dyes, or any combination thereof.

In some embodiments, the exposing is performed using a mirror, a mirror array, a lens, a moving stage, or a photomask. In some embodiments, the exposing is performed using a scanning laser. In some embodiments, the wavelength of light is about 100 nm to about 600 nm. In some embodiments, the wavelength of light is about 250 nm to about 400 nm. In some embodiments, the wavelength of light is about 300 nm to about 350 nm.

In some embodiments, removing the cleaved capture probes comprises washing.

In some embodiments, the method further comprises permeabilizing the biological sample. In some embodiments, permeabilizing the biological sample is performed before contacting the analyte with the capture domain of the capture probe in the area of the array covered by the biological sample. In some embodiments, the method further comprises extending a 3′ end of the capture probe in the area of the array covered by the biological sample using the analyte as an extension template.

In some embodiments, the determining in step (e) comprises sequencing (i) the spatial barcode of the capture probe or a complement thereof in the area of the array covered by the biological sample, and (ii) all or a portion of the analyte bound to the capture domain of the capture probe or a complement thereof in the area of the array covered by the biological sample. In some embodiments, the sequencing is high throughput sequencing.

In some embodiments, the analyte/analyte(s) is/are DNA. In some embodiments, the DNA is genomic DNA. In some embodiments, the analyte(s) is/are RNA. In some embodiments, the RNA is mRNA. In some embodiments, the analyte(s) is/are ligation product(s).

In some embodiments, the method further comprises, contacting a nucleic acid in the biological sample with a first probe and a second probe, wherein the first probe comprises a sequence that hybridizes to a first sequence in the nucleic acid in the biological sample, and the second probe comprises (i) a sequence that hybridizes to a second sequence in the nucleic acid in the biological sample, and (ii) a sequence that binds the capture domain of the capture probe in the area of the array covered by the biological sample; and ligating the first probe and the second probe to generate the ligation product, wherein the ligation product hybridizes to the capture domain of the capture probe in the area of the array covered by the biological sample.

In some embodiments, the biological sample is a tissue sample. In some embodiments, the tissue sample is a tissue section. In some embodiments, the tissue section is a fresh, frozen tissue section. In some embodiments, the tissue section is a fixed tissue section. In some embodiments, the array comprises one or more features. In some embodiments, the one or more features comprise a bead.

In another aspect, provided herein is a method for determining a location of an analyte in a biological sample, the method comprising (a) contacting a biological sample with an array comprising a plurality of capture probes, wherein each capture probe of the plurality of capture probes comprises: (i) a photocleavable linker; (ii) a spatial barcode, and (iii) a capture domain; (b) contacting a plurality of analyte capture agents with the biological sample comprising (i) an analyte capture sequence that hybridizes to the capture domains of the plurality of capture probes, (ii) an analyte binding moiety barcode, and (iii) an analyte binding moiety that binds the analyte; (c) exposing the area of the array not covered by the biological sample with a wavelength of light that cleaves the photocleavable linker of the capture probes, thereby releasing the capture probes from the area of the array not covered by the biological sample; (d) removing the cleaved capture probes; (e) contacting an analyte capture agent in the biological sample with a capture domain of a capture probe in an area of the array covered by the biological sample; and (f) determining (i) the sequence of the spatial barcode of the capture probe or a complement thereof in the area of the array covered by the biological sample, and (ii) all or a portion of the sequence of the analyte binding moiety barcode, or a complement thereof, and using the sequences of (i) and (ii) to determine the location of the analyte in the biological sample.

In another aspect, provided herein are methods of decreasing hybridization of analyte capture agents to capture probes in an area of an array that is not covered by the biological sample that include: (a) contacting the biological sample with the array, wherein the array comprises a plurality of capture probes comprising (i) a photocleavable linker; (ii) a spatial barcode, and (iii) a capture domain; (b) contacting the analyte capture agents with the biological sample, wherein the analyte capture agents comprise (i) an analyte capture sequence that hybridizes to the capture domains of the plurality of capture probes, (ii) an analyte binding moiety barcode, and (iii) an analyte binding moiety that binds to an analyte; and (c) exposing the area of the array that is not covered by the biological sample with a wavelength of light that cleaves the photocleavable linker of the capture probes, thereby releasing the capture probes from the area of the array not covered by the biological sample, thereby decreasing hybridization of analyte capture agents to capture probes in the area of the array that is not covered by the biological sample.

In some embodiments, wherein the method further comprises, prior to step (c), imaging the biological sample on the array. In some embodiments, the imaging is performed using expansion microscopy, bright field microscopy, dark field microscopy, phase contrast microscopy, electron microscopy, fluorescence microscopy, reflection microscopy, interference microscopy, confocal microscopy, and visual identification, or any combination thereof. In some embodiments, the imaging is performed using phase contrast microscopy.

In some embodiments, the method further comprises staining the biological sample. In some embodiments, the staining comprises the use of acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or any combination thereof. In some embodiments, the staining comprises the use of eosin and hematoxylin. In some embodiments, the staining comprises the use of radioisotopes, fluorophores, chemiluminescent compounds, bioluminescent compounds, dyes, or any combination thereof.

In some embodiments, the exposing is performed using a mirror, a mirror array, a lens, a moving stage, or a mask. In some embodiments, the exposing is performed using a scanning laser. In some embodiments, the wavelength of light is about 100 nm to about 600 nm. In some embodiments, the wavelength of light is about 250 nm to about 400 nm. In some embodiments, the wavelength of light is about 300 nm to about 350 nm. In some embodiments, the wavelength of light is about 300 nm to about 350 nm.

In some embodiments, removing the cleaved capture probes comprises washing.

In some embodiments, the method further comprises permeabilizing the biological sample. In some embodiments, the step of permeabilizing the biological sample is performed before contacting the analyte capture agent with the capture domain of the capture probe in the area of the array covered by the biological sample. In some embodiments, the method further comprises extending a 3′ end of the capture probe in the area of the array covered by the biological sample using the analyte capture agent as an extension template.

In some embodiments, the determining in step (e) comprises sequencing (i) the spatial barcode of the capture probe or a complement thereof in the area of the array covered by the biological sample, and (ii) all or a portion of the analyte binding moiety barcode, or a complement thereof. In some embodiments, the sequencing is high throughput sequencing. In some embodiments, the analyte is a protein. In some embodiments, the protein is an intracellular protein. In some embodiments, the protein is an extracellular protein.

In some embodiments, the analyte binding moiety is an antibody or an antigen-binding fragment thereof. In some embodiments, steps (a) and (b) are performed at substantially the same time. In some embodiments, step (a) is performed before step (b). In some embodiments, step (b) is performed before step (a).

In some embodiments, wherein the biological sample is a tissue sample. In some embodiments, the tissue sample is a tissue section. In some embodiments, the tissue section is a fresh, frozen tissue section. In some embodiments, the tissue section is a fixed tissue section. In some embodiments, the array comprises one or more features. In some embodiments, the one or more features comprises a bead.

In another aspect, provided herein are methods for determining a location of an analyte in a biological sample that include: (a) contacting a biological sample with an array comprising a plurality of capture probes, wherein each capture probe of the plurality of capture probes comprises (i) a capture domain, (ii) a cleavage sequence, and (iii) a spatial barcode, wherein the cleavage sequence is positioned 5′ relative to the capture domain and the 5′ end of the capture probe is attached to a substrate; (b) contacting the array with a plurality of cleavage probes, wherein a cleavage probe of the plurality of cleavage probes hybridizes to a cleavage sequence in a capture probe in an area of the array not covered by the biological sample; (c) contacting the area of the array not covered by the biological sample with an enzyme that cleaves the cleavage sequence hybridized to the cleavage probe, thereby releasing the capture probe from the area of the array not covered by the biological sample; (d) removing the cleaved capture probe and the enzyme from the array; (e) contacting an analyte in the biological sample with a capture domain of a capture probe in an area of the array covered by the biological sample; and (f) determining (i) the sequence of the spatial barcode of the capture probe or a complement thereof in the area of the array covered by the biological sample, and (ii) all or a portion of the sequence of the analyte bound to the capture domain of the capture probe or a complement thereof in the area of the array covered by the biological sample, and using the sequences of (i) and (ii) to determine the location of the analyte in the biological sample.

In another aspect, provided herein are methods of decreasing hybridization of analytes in a biological sample to capture probes in an area of an array that is not covered by the biological sample that include: (a) contacting the biological sample with the array, wherein the array comprises a plurality of capture probes, wherein each capture probe of the plurality of capture probes comprises (i) a capture domain, (ii) a cleavage sequence, and (iii) a spatial barcode, wherein the cleavage sequence is positioned 5′ relative to the capture domain and the 5′ end of the capture probe is attached to a substrate; (b) contacting the array with a plurality of cleavage probes, wherein a cleavage probe of the plurality of cleavage probes hybridizes to a cleavage sequence in a capture probe in an area of the array not covered by the biological sample; and (c) contacting the area of the array not covered by the biological sample with an enzyme that cleaves the cleavage sequence hybridized to the cleavage probe, thereby releasing the capture probe from the area of the array not covered by the biological sample, thereby decreasing hybridization of analytes in the biological sample to capture probes in the area of the array that is not covered by the biological sample..

In some embodiments, the spatial barcode is positioned 3′ to the cleavage sequence. In some embodiments, the spatial barcode is positioned 5′ to the cleavage sequence. In some embodiments, the array comprises a plurality of subpopulations of capture probes, wherein each subpopulation of the plurality of subpopulations of capture probes comprises a unique cleavage sequence. In some embodiments, each subpopulation of capture probes is arranged in a geometric pattern on the array. In some embodiments, each subpopulation of capture probes is arranged in a concentric pattern on the array.

In some embodiments, the hybridization of the cleavage sequence to the cleavage probe results in the formation of a cleavage site comprising DNA, RNA, or a combination thereof. In some embodiments, the hybridization of the cleavage sequence to the cleavage probe results in the formation of a restriction endonuclease cleavage site or a CRISPR/Cas cleavage site. In some embodiments, the enzyme is a DNase, an RNase, a restriction endonuclease, and/or a Cas enzyme. In some embodiments, the capture probes are cleaved in their entirety or in part from the area of the array not covered by the biological sample.

In some embodiments, the method further comprises, prior to step (c), imaging the biological sample on the array. In some embodiments, the imaging is performed using expansion microscopy, bright field microscopy, dark field microscopy, phase contrast microscopy, electron microscopy, fluorescence microscopy, reflection microscopy, interference microscopy, confocal microscopy, and visual identification, or any combination thereof. In some embodiments, the imaging is performed using phase contrast microscopy.

In some embodiments, the method further comprises, prior to step (c), staining the biological sample. In some embodiments, the staining comprises use of acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or any combination thereof. In some embodiments, the staining comprises the use of eosin and hematoxylin. In some embodiments, the staining comprises use of radioisotopes, fluorophores, chemiluminescent compounds, bioluminescent compounds, dyes, or any combination thereof.

In some embodiments, the method further comprises between steps (b) and (c), removing the unhybridized cleavage probes from the array. In some embodiments, the step of removing the unhybridized capture probes from the array comprises washing. In some embodiments, the step of removing the cleaved capture probe and the enzyme from the array comprises washing.

In some embodiments, the method further comprises permeabilizing the biological sample after step (d). In some embodiments, the method further comprises extending a 3′ end of the capture probe using the analyte as an extension template.

In some embodiments, the determining in step (g) comprises sequencing (i) the spatial barcode of the capture probe in the area of the array covered by the biological sample, or a complement thereof, and (ii) all or a portion of the analyte bound to the capture domain of the capture probe in the area of the array covered by the biological sample, or a complement thereof. In some embodiments, the sequencing is high throughput sequencing.

In some embodiments, the analyte(s) is/are DNA. In some embodiments, the DNA is genomic DNA. In some embodiments, the analyte(s) is/are RNA. In some embodiments, the RNA is mRNA.

In some embodiments, the analyte(s) is/are ligation product(s). In some embodiments, the method further comprises, contacting a nucleic acid in the biological sample with a first probe and a second probe, wherein the first probe comprises a sequence that hybridizes to a first sequence in the nucleic acid in the biological sample, and the second probe comprises (i) a sequence that hybridizes to a second sequence in the nucleic acid in the biological sample, and (ii) a sequence that binds the capture domain of the capture probe in the area of the array covered by the biological sample; and ligating the first probe and the second probe to generate the ligation product, wherein the ligation product hybridizes to the capture domain of the capture probe in the area of the array covered by the biological sample.

In some embodiments, the biological sample is a tissue sample. In some embodiments, the tissue sample is a tissue section. In some embodiments, the tissue section is a fresh, frozen tissue section. In some embodiments, the tissue section is a fixed tissue section. In some embodiments, the array comprises one or more features. In some embodiments, the one or more features comprise a bead.

In another aspect, provided herein are methods for determining a location of an analyte in a biological sample that include: (a) contacting a biological sample with an array comprising a plurality of capture probes, wherein each capture probe of the plurality of capture probes comprises (i) a capture domain, (ii) a cleavage sequence, and (iii) a spatial barcode, wherein the cleavage sequence is positioned 5′ relative to the capture domain and the 5′ end of the capture probe is attached to a substrate; (b) contacting a plurality of analyte capture agents with the biological sample, wherein each analyte capture agent of the plurality of analyte capture agents comprises (i) an analyte capture sequence that hybridizes to the capture domains of the plurality of capture probes, (ii) an analyte binding moiety barcode, and (iii) an analyte binding moiety that binds the analyte; (c) contacting the array with a plurality of cleavage probes, wherein a cleavage probe of the plurality of cleavage probes hybridizes to a cleavage sequence in a capture probe in an area of the array not covered by the biological sample; (d) contacting the area of the array not covered by the biological sample with an enzyme that cleaves the cleavage sequence hybridized to the cleavage probe, thereby releasing the capture probe from the area of the array not covered by the biological sample; (e) removing the cleaved capture probe and the enzyme from the array; (f) contacting an analyte capture agent in the biological sample with a capture domain of a capture probe in an area of the array covered by the biological sample; and (g) determining (i) the sequence of the spatial barcode of the capture probe in the area of the array covered by the biological sample, or a complement thereof, and (ii) all or a portion of the sequence of the analyte binding moiety barcode, or a complement thereof, and using the sequences of (i) and (ii) to determine the location of the analyte in the biological sample.

In another aspect, provided herein are methods of decreasing hybridization of analyte capture agents to capture probes in an area of an array that is not covered by the biological sample that include: (a) contacting the biological sample with the array, wherein the array comprises a plurality of capture probes, wherein each capture probe of the plurality of capture probes comprises (i) a capture domain, (ii) a cleavage sequence, and (iii) a spatial barcode, wherein the cleavage sequence is positioned 5′ relative to the capture domain and the 5′ end of the capture probe is attached to a substrate; (b) contacting a plurality of analyte capture agents with the biological sample, wherein each analyte capture agent of the plurality of analyte capture agents comprises (i) an analyte capture sequence that hybridizes to the capture domains of the plurality of capture probes, (ii) an analyte binding moiety barcode, and (iii) an analyte binding moiety that binds the analyte; (c) contacting the array with a plurality of cleavage probes, wherein a cleavage probe of the plurality of cleavage probes hybridizes to a cleavage sequence in a capture probe in an area of the array not covered by the biological sample; and (d) contacting the area of the array not covered by the biological sample with an enzyme that cleaves the cleavage sequence hybridized to the cleavage probe, thereby releasing the capture probe from the area of the array not covered by the biological sample, thereby decreasing hybridization of analyte capture agents to capture probes in the area of the array that is not covered by the biological sample.

In some embodiments, the spatial barcode is positioned 3′ to the cleavage sequence. In some embodiments, the spatial barcode is positioned 5′ to the cleavage sequence.

In some embodiments, the array comprises a plurality of subpopulations of capture probes, wherein each subpopulation of the plurality of subpopulations of capture probes comprises a unique cleavage sequence. In some embodiments, each subpopulation of capture probes is arranged in a geometric pattern on the array. In some embodiments, each subpopulation of capture probes is arranged in a concentric pattern on the array.

In some embodiments, the hybridization of the cleavage sequence to the cleavage probe results in the formation of a cleavage site comprising DNA, RNA, or a combination thereof. In some embodiments, the hybridization of the cleavage sequence to the cleavage probe results in the formation of a restriction endonuclease cleavage site or a CRISPR/Cas cleavage site. In some embodiments, the enzyme is a DNase, an RNase, a restriction endonuclease, and/or a Cas enzyme. In some embodiments, the capture probes are cleaved in their entirety or in part from the area of the array not covered by the biological sample.

In some embodiments, the method further comprises, prior to step (d), imaging the biological sample on the array. In some embodiments, the imaging is performed using expansion microscopy, bright field microscopy, dark field microscopy, phase contrast microscopy, electron microscopy, fluorescence microscopy, reflection microscopy, interference microscopy, confocal microscopy, and visual identification, or any combination thereof. In some embodiments, the imaging is performed using phase contrast microscopy.

In some embodiments, the method further comprises, prior to step (d), staining the biological sample. In some embodiments, the staining comprises use of acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or any combination thereof. In some embodiments, the staining comprises use of eosin and hematoxylin. In some embodiments, the staining comprises use of radioisotopes, fluorophores, chemiluminescent compounds, bioluminescent compounds, dyes, or any combination thereof.

In some embodiments, the method further comprises between steps (c) and (d), removing the unhybridized cleavage probes from the array. In some embodiments, the step of removing the unhybridized capture probes from the array comprises washing. In some embodiments, the step of removing the cleaved capture probe and the enzyme from the array comprises washing.

In some embodiments, the method further comprises permeabilizing the biological sample after step (e). In some embodiments, the method further comprises extending a 3′ end of the capture probe using the analyte capture agent as an extension template.

In some embodiments, the determining in step (g) comprises sequencing (i) the spatial barcode of the capture probe in the area of the array covered by the biological sample, or a complement thereof, and (ii) all or a portion of the analyte binding moiety barcode, or a complement thereof. In some embodiments, the sequencing is high throughput sequencing.

In some embodiments, the analyte(s) is/are protein(s). In some embodiments, the protein(s) is/are intracellular protein(s). In some embodiments, the protein(s) is/are extracellular protein(s). In some embodiments, the analyte binding moiety is an antibody or an antigen-binding fragment thereof.

In some embodiments, steps (a) and (b) are performed at substantially the same time. In some embodiments, step (a) is performed before step (b). In some embodiments, step (b) is performed before step (a).

In some embodiments, the biological sample is a tissue sample. In some embodiments, the tissue sample is a tissue section. In some embodiments, the tissue section is a fresh, frozen tissue section. In some embodiments, the tissue section is a fixed tissue section.

In some embodiments, the array comprises one or more features. In some embodiments, the one or more features comprises a bead.

In another aspect, provided herein are systems that include: a light source configured to generate light; an optical assembly configured to direct the generated light to a plurality of different spatial locations; a fluid handling assembly comprising a fluid reservoir, a waste reservoir, and one or more fluid channels; a stage configured to receive a substrate comprising an array of features and a tissue sample positioned on a subset of the array of features; and a controller coupled to the light source, the optical assembly, and the fluid handling assembly, and configured to: control the optical assembly to selectively expose features of the array that are not members of the subset of features to the generated light to cleave capture probes from the exposed features; and control the fluid handling assembly to deliver a fluid from the fluid reservoir to the array to remove the cleaved capture probes from the array and transport the cleaved capture probes to the waste reservoir.

In some embodiments, the controller is configured to identify the features of the array that are not members of the subset of features. In some embodiments, the system further comprises a detector coupled to the controller, wherein the controller is configured use the detector to obtain an image of the tissue sample positioned on the subset of the array of features, and to identify the features of the array that are not members of the subset of features based on the image. In some embodiments, the optical assembly comprises one or more mirrors coupled to the controller, and wherein the controller is configured to adjust at least one of an orientation and position of the one or more mirrors to selectively expose the features of the array that are not members of the subset of features to the generated light. In some embodiments, the optical assembly comprises a mask coupled to the controller, and wherein the controller is configured to adjust the mask to selectively expose the features of the array that are not members of the subset of features to the generated light.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 shows an exemplary example of a photocleavable capture probe.

FIGS. 2A-E shows an exemplary workflow for cleaving capture probes around a biological sample (e.g., not covered by the biological sample).

FIG. 3A shows exemplary capture probes comprising photocleavable groups on the surface of a slide.

FIG. 3B shows an exemplary workflow for cleaving capture probes in an area of the array not covered by the biological sample.

FIGS. 4A-D show an exemplary workflow for cleaving capture probes comprising a cleavage sequence on an array using cleavage probes and an enzyme.

FIG. 5A-B shows an exemplary area of the array covered by a biological sample after cleavage of the capture probes in an area of the array not covered by the biological sample.

FIG. 6A is a schematic diagram showing an example sample handling apparatus.

FIG. 6B is a schematic diagram showing an example imaging apparatus.

FIG. 6C is a schematic diagram showing one example of a control unit.

### DETAILED DESCRIPTION

An array can include multiple capture probes coupled to a substrate. The capture probes can include a linker, a spatial barcode, and a capture domain. The spatial barcode can spatially-resolve a molecular component (e.g., an analyte) found in the biological sample. In some examples, spatial barcodes can spatially-resolve molecular components at single-cell resolution. For instance, a biological sample can be placed on the array for an assay that is targeting analytes within the biological sample, and the spatial barcode can provide a location of the analyte in the biological sample. In some incidences, a target analyte and/or an intermediate agent from the biological sample can bind to capture probes outside of the biological sample (e.g., not covered by the biological sample). For example, during an assay (e.g., a permeabilization step, a staining step, a washing step, etc.), an analyte can migrate from the biological sample and be captured by a capture probe outside of the location where the biological sample is positioned. Analytes binding to capture probes outside of the location where the biological sample is positioned can result in skewed results via producing sequencing reads from areas of the array that are not covered by the biological sample. The sequencing reads from areas not covered by the biological sample are discarded, causing a waste of resources and time.

In other incidences, assay reagents such as probes can bind to capture probes in areas not covered by the biological sample. Errant binding of reagents such as primers or probes, to capture probes in areas not covered by the biological sample, can decrease the sensitivity of an assay. For example, during an assay, primers or probes intended to bind to a target analyte of the biological sample can errantly bind to capture probes in areas not covered by the biological sample. This can decrease the sensitivity of the assay by decreasing the number of probes available to bind to the intended target analyte of the biological sample.

The capture probes of the array can attach to the substrate of the array using many different methods. For example, capture probes can have a variety of linkers that are detachable from the substrate under appropriate conditions. Detachment can be achieved through physical, chemical, or enzymatic means depending on the type of linkers that are used. These cleavable capture probes can be selectively removed from the substrate at determined locations. Among these cleavable linkers, photocleavable linkers are particularly attractive as they can be rapidly cleaved from the substrate. They can also be cleaved with high accuracy, e.g., by masking or by directed UV light (e.g., a UV laser).

As described herein, a biological sample can be positioned on an array with capture probes including photocleavable linkers. The capture probes located in an area not covered by the biological sample can be removed from the substrate by exposing the area of the array not covered by the biological sample with a wavelength of light. In this way, the capture probes remaining on the array area located under the biological sample remain intact and able to capture analytes from the biological sample whereas those around the biological sample are not able to capture analytes. This can increase the speed and efficiency of the assay by permitting only the capture probes located under the biological sample to bind to analytes and/or interact with reagents such as primers or probes. The cleaved capture probes can be removed from the array through washing and the assay can proceed.

Provided herein are methods of cleaving the capture probes that are located on a portion or area of an array that is not covered by the biological sample. In some cases, capture probes can comprise a photocleavable linker. In some cases, the area of the array not covered by the biological sample can be contacted with light to cleave the photocleavable linker of one or more of the capture probes located in an area not covered by the biological sample.

In other embodiments, the capture probes comprise a cleavage sequence, a spatial barcode, and a capture domain, and the area of the array not covered by the biological sample is contacted with a cleavage probe that can hybridize to the cleavage sequence and an enzyme that cleaves the cleavage sequence/cleavage probe hybrid. The cleaved capture probes can be removed from the array through washing and the assay can proceed. In some embodiments, the cleavage sequence is the same for every capture probe. In other embodiments, the cleavage sequence can be different in one or more areas of the array such that in one area of the array the cleavage sequence is the same comparative to a second area of the array, or a third area, or a fourth area, etc. In some embodiments, the cleavage sequence is unique to each capture probe on the array. In this manner, one or more than one method for cleaving the cleavage sequence/cleavage probe hybrid can be implemented as desired.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell or a tissue sample. Spatial analysis methods and compositions can also include the use of a capture probe having a capture domain that captures an intermediate agent for indirect detection of an analyte. For example, the intermediate agent can include a nucleic acid sequence (e.g., an analyte binding moiety barcode) associated with the intermediate agent (e.g., an analyte capture agent). Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 10 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest. An analyte binding moiety barcode can be used to spatially identify one analyte from another as captured from a biological sample.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier). In some embodiments, a capture probe can include a photocleavable linker or a cleavage sequence and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663. In some embodiments, a capture probe can comprise a photocleavable linker, a spatial barcode, and a capture domain. In some embodiments, a capture probe can comprise a spatial barcode, a cleavage sequence, and a capture domain.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

A “cleavage sequence” refers to a sequence in a capture probe that is capable of hybridizing to a cleavage probe, where an enzyme is capable of cleaving the cleavage sequence upon its hybridization with the cleavage probe. Likewise, a “cleavage probe” is a nucleic acid molecule that is capable of hybridizing to a cleavage sequence in a capture probe, where an enzyme is capable of cleaving the cleavage sequence in the capture probe upon its hybridization with the cleavage probe. In some embodiments, the cleavage sequence and/or the cleavage probe comprise about 10 nucleotides to about 50 nucleotides, about 10 nucleotides to about 40 nucleotides, or about 10 nucleotides to about 30 nucleotides.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes to migrate towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent (e.g., an analyte binding moiety barcode of an analyte capture agent) bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extending the capture probe includes adding to the 3′ end of a capture probe a nucleic acid sequence that is complementary to the analyte binding moiety barcode of an analyte capture agent specifically bound to the capture domain of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### Cleaving Capture Probes in an Area not Covered by a Biological Sample

As described herein, analytes binding to capture probes in an area not covered by the biological sample can result in skewed results via producing sequencing reads from areas of the array not covered by the biological sample. The sequencing reads from areas not covered by the biological sample are discarded, resulting in a waste of resources and time. Alternatively, assay reagents such as probes can bind to capture probes in areas not covered by the biological sample. Errant binding of reagents such as probes, to capture probes in areas not covered by the biological sample, can decrease the sensitivity of an assay. For example, during an assay, probes intended to bind to a target of the biological sample can errantly bind to capture probes in areas not covered by the biological sample. This can decrease the sensitivity of the assay by decreasing the amount of probes available to bind to the intended target of the biological sample. Thus, the present disclosure features methods to remove (e.g., cleave) capture probes in areas not covered by the biological sample.

For the spatial array-based analytical methods described herein, a substrate functions as a support for direct or indirect attachment of capture probes to features of the array. In addition, in some embodiments, a substrate (e.g., the same substrate or a different substrate) can be used to provide support to a biological sample, particularly, for example, a tissue section. Accordingly, a “substrate” is a support that is insoluble in aqueous liquid and which allows for positioning of biological samples, analytes, features, and/or capture probes on the substrate.

Further, a “substrate” as used herein, and when not preceded by the modifier “chemical”, refers to a member with at least one surface that generally functions to provide physical support for biological samples, analytes, and/or any of the other chemical and/or physical moieties, agents, and structures described herein. Substrates can be formed from a variety of solid materials, gel-based materials, colloidal materials, semi-solid materials (e.g., materials that are at least partially cross-linked), materials that are fully or partially cured, and materials that undergo a phase change or transition to provide physical support. Examples of substrates that can be used in the methods and systems described herein include, but are not limited to, slides (e.g., slides formed from various glasses, slides formed from various polymers), hydrogels, layers and/or films, membranes (e.g., porous membranes), flow cells, cuvettes, wafers, plates, or combinations thereof. In some embodiments, substrates can optionally include functional elements such as recesses, protruding structures, microfluidic elements (e.g., channels, reservoirs, electrodes, valves, seals), and various markings.

In some embodiments, the capture probe is a conjugate (e.g., an oligonucleotide-antibody conjugate). In some embodiments, the capture probe includes a barcode (e.g., a spatial barcode and/or a unique molecular identifier (UMI)), and a photocleavable linker and/or a cleavage sequence, and a capture domain.

The capture probe is optionally coupled to a feature by a cleavage domain, such as a disulfide linker, a photocleavable linker, a cleavage site, or a cleavage sequence. The capture probe can include functional sequences that are useful for subsequent processing, such as a sequencer specific flow cell attachment sequence, e.g., a P5 or P7 sequence, as well as functional sequences such as sequencing primer sequences, e.g., a R1 primer binding site, a R2 primer binding site. In some embodiments, a sequence is a P7 sequence and a sequence is a R2 primer binding site. A spatial barcode can be included within the capture probe for use in barcoding the target analyte for identifying its relative spatial location in a biological sample. The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of commercialized sequencing systems and techniques, for which suitable functional sequences can be used, include (but are not limited to) Ion Torrent Proton or PGM sequencing, Illumina sequencing, PacBio SMRT sequencing, and Oxford Nanopore sequencing.

In some embodiments, the spatial barcode, and functional sequences such as flow cell attachment sequences and sequencing primer sequences can be common to all of the probes attached to a given feature. In some embodiments, the cleavage sequence can be common to all of the probes attached to a given feature. The capture probe can include a capture domain to facilitate capture of a target analyte, a ligation product, or an analyte capture agent.

Each capture probe includes at least one capture domain. The “capture domain” can be an oligonucleotide, a polypeptide, a small molecule, or any combination thereof, that hybridizes or hybridizes specifically to a desired analyte, ligation product, or analyte capture agent. In some embodiments, a capture domain can be used to capture or detect a desired analyte.

In some embodiments, the capture domain is a functional nucleic acid sequence configured to interact with one or more analytes, such as one or more different types of nucleic acids (e.g., RNA molecules and DNA molecules). In some embodiments, the capture domain can include an N-mer sequence (e.g., a random N-mer sequence), which N-mer sequences are configured to interact with a plurality of DNA molecules. In some embodiments, the capture domain can include a poly(T) sequence, which poly(T) sequences are configured to interact with messenger RNA (mRNA) molecules via the poly(A) tail of an mRNA transcript. In some embodiments, the capture domain is the binding target of a protein (e.g., a transcription factor, a DNA binding protein, or a RNA binding protein), where the analyte of interest is a protein.

Capture probes can include ribonucleotides and/or deoxyribonucleotides as well as synthetic nucleotide residues that are capable of participating in Watson-Crick type or analogous base pair interactions. In some embodiments, the capture domain is capable of priming a reverse transcription reaction to generate cDNA that is complementary to the captured RNA molecules. In some embodiments, the capture domain of the capture probe can prime a DNA extension (polymerase) reaction to generate DNA that is complementary to the captured DNA molecules. In some embodiments, the capture domain can template a ligation reaction between the captured DNA molecules and a surface probe that is directly or indirectly immobilized on the substrate. In some embodiments, the capture domain can be ligated to one strand of the captured DNA molecules. For example, SplintR ligase along with RNA or DNA sequences (e.g., degenerate RNA) can be used to ligate a single-stranded DNA or RNA to the capture domain. In some embodiments, ligases with RNA-templated ligase activity, e.g., SplintR ligase, T4 RNA ligase 2 or KOD ligase, can be used to ligate a single-stranded DNA or RNA to the capture domain. In some embodiments, a capture domain includes a sequence that will hybridize to a splint oligonucleotide. In some embodiments, a capture domain captures a splint oligonucleotide.

In some embodiments, the capture domain is located at the 3′ end of the capture probe and includes a free 3′ end that can be extended, e.g., by template dependent polymerization, to form an extended capture probe as described herein. In some embodiments, the capture domain includes a nucleotide sequence that is capable of hybridizing to a nucleic acid, e.g., RNA or other analyte, present in the cells of the biological sample contacted with the array. In some embodiments, the capture domain can be selected or designed to hybridize selectively or specifically to a target nucleic acid. For example, the capture domain can be selected or designed to capture mRNA by way of hybridization to the mRNA poly(A) tail. Thus, in some embodiments, the capture domain includes a poly(T) DNA oligonucleotide, e.g., a series of consecutive deoxythymidine residues linked by phosphodiester bonds, which is capable of hybridizing to the poly(A) tail of mRNA. In some embodiments, the capture domain can include nucleotides that are functionally or structurally analogous to a poly(T) tail. For example, a poly(U) oligonucleotide or an oligonucleotide included of deoxythymidine analogues. In some embodiments, the capture domain includes at least 10, 11, 12, 13, 14, 15, 16, 17, 18, 19, or 20 nucleotides. In some embodiments, the capture domain includes at least 25, 30, or 35 nucleotides.

In some embodiments, a capture probe includes a capture domain having a sequence that is capable of hybridizing to mRNA and/or genomic DNA. For example, the capture probe can include a capture domain that includes a nucleic acid sequence (e.g., a poly(T) sequence) capable of binding to a poly(A) tail of an mRNA and/or to a poly(A) homopolymeric sequence present in genomic DNA. In some embodiments, a homopolymeric sequence is added to an mRNA molecule or a genomic DNA molecule using a terminal transferase enzyme in order to produce an analyte that has a poly(A) or poly(T) sequence. For example, a poly(A) sequence can be added to an analyte (e.g., a fragment of genomic DNA) thereby making the analyte capable of capture by a poly(T) capture domain.

In some embodiments, random sequences, e.g., random hexamers or similar sequences, can be used to form all or a part of the capture domain. For example, random sequences can be used in conjunction with poly(T) (or poly(T) analogue) sequences. Thus, where a capture domain includes a poly(T) (or a “poly(T)-like”) oligonucleotide, it can also include a random oligonucleotide sequence (e.g., “poly(T)-random sequence” probe). This can, for example, be located 5′ or 3′ of the poly(T) sequence, e.g., at the 3′ end of the capture domain. The poly(T)-random sequence probe can facilitate the capture of the mRNA poly(A) tail. In some embodiments, the capture domain can be an entirely random sequence. In some embodiments, degenerate capture domains can be used.

In some embodiments, a pool of two or more capture probes form a mixture, where the capture domain of one or more capture probes includes a poly(T) sequence and the capture domain of one or more capture probes includes random sequences. In some embodiments, a pool of two or more capture probes form a mixture where the capture domain of one or more capture probes includes poly(T)-like sequence and the capture domain of one or more capture probes includes random sequences. In some embodiments, a pool of two or more capture probes form a mixture where the capture domain of one or more capture probes includes a poly(T)-random sequences and the capture domain of one or more capture probes includes random sequences. In some embodiments, probes with degenerate capture domains can be added to any of the preceding combinations listed herein. In some embodiments, probes with degenerate capture domains can be substituted for one of the probes in each of the pairs described herein.

The capture domain can be based on a particular gene sequence or particular motif sequence or common/conserved sequence, that it is designed to capture (i.e., a sequence-specific capture domain). Thus, in some embodiments, the capture domain is capable of binding selectively to a desired sub-type or subset of nucleic acid, for example a particular type of RNA, such as mRNA, rRNA, tRNA, SRP RNA, tmRNA, snRNA, snoRNA, SmY RNA, scaRNA, gRNA, RNase P, RNase MRP, TERC, SL RNA, aRNA, cis-NAT, crRNA, lncRNA, miRNA, piRNA, siRNA, shRNA, tasiRNA, rasiRNA, 7SK, eRNA, ncRNA or other types of RNA. In a non-limiting example, the capture domain can be capable of binding selectively to a desired subset of ribonucleic acids, for example, microbiome RNA, such as 16S rRNA.

In some embodiments, a capture domain includes an “anchor” or “anchoring sequence”, which is a sequence of nucleotides that is designed to ensure that the capture domain hybridizes to the intended analyte. In some embodiments, an anchor sequence includes a sequence of nucleotides, including a 1-mer, 2-mer, 3-mer or longer sequence. In some embodiments, the short sequence is random. For example, a capture domain including a poly(T) sequence can be designed to capture an mRNA. In such embodiments, an anchoring sequence can include a random 3-mer (e.g., GGG) that helps ensure that the poly(T) capture domain hybridizes to an mRNA. In some embodiments, an anchoring sequence can be VN, N, or NN. Alternatively, the sequence can be designed using a specific sequence of nucleotides. In some embodiments, the anchor sequence is at the 3′ end of the capture domain. In some embodiments, the anchor sequence is at the 5′ end of the capture domain.

In some embodiments, capture domains of capture probes are blocked prior to contacting the biological sample with the array, and blocking probes are used when the nucleic acid in the biological sample is modified prior to its capture on the array. In some embodiments, the blocking probe is used to block or modify the free 3′ end of the capture domain. In some embodiments, blocking probes can be hybridized to the capture probes to mask the free 3′ end of the capture domain, e.g., hairpin probes, partially double stranded probes, or complementary sequences. In some embodiments, the free 3′ end of the capture domain can be blocked by chemical modification, e.g., addition of an azidomethyl group as a chemically reversible capping moiety such that the capture probes do not include a free 3′ end. Blocking or modifying the capture probes, particularly at the free 3′ end of the capture domain, prior to contacting the biological sample with the array, prevents modification of the capture probes, e.g., prevents the addition of a poly(A) tail to the free 3′ end of the capture probes.

Non-limiting examples of 3′ modifications include dideoxy C-3′ (3′-ddC), 3′ inverted dT, 3′ C3 spacer, 3′Amino, and 3′ phosphorylation. In some embodiments, the nucleic acid in the biological sample can be modified such that it can be captured by the capture domain. For example, an adaptor sequence (including a binding domain capable of binding to the capture domain of the capture probe) can be added to the end of the nucleic acid, e.g., fragmented genomic DNA. In some embodiments, this is achieved by ligation of the adaptor sequence or extension of the nucleic acid. In some embodiments, an enzyme is used to incorporate additional nucleotides at the end of the nucleic acid sequence, e.g., a poly(A) tail. In some embodiments, the capture probes can be reversibly masked or modified such that the capture domain of the capture probe does not include a free 3′ end. In some embodiments, the 3′ end is removed, modified, or made inaccessible so that the capture domain is not susceptible to the process used to modify the nucleic acid of the biological sample, e.g., ligation or extension.

In some embodiments, the capture domain of the capture probe is modified to allow the removal of any modifications of the capture probe that occur during modification of the nucleic acid molecules of the biological sample. In some embodiments, the capture probes can include an additional sequence downstream of the capture domain, e.g., 3′ to the capture domain, namely a blocking domain.

In some embodiments, the capture domain of the capture probe can be a non-nucleic acid domain. Examples of suitable capture domains that are not exclusively nucleic-acid based include, but are not limited to, proteins, peptides, aptamers, antigens, antibodies, and molecular analogs that mimic the functionality of any of the capture domains described herein.

Each capture probe can optionally include at least one functional domain. Each functional domain typically includes a functional nucleotide sequence for a downstream analytical step in the overall analysis procedure.

In some embodiments, the capture probe can include a functional domain for attachment to a sequencing flow cell, such as, for example, a P5 sequence for Illumina® sequencing. In some embodiments, the capture probe or derivative thereof can include another functional domain, such as, for example, a P7 sequence for attachment to a sequencing flow cell for Illumina® sequencing. The functional domains can be selected for compatibility with a variety of different sequencing systems, e.g., 454 Sequencing, Ion Torrent Proton or PGM, Illumina, etc., and the requirements thereof.

In some embodiments, the functional domain includes a primer. The primer can include an R1 primer sequence for Illumina® sequencing, and in some embodiments, an R2 primer sequence for Illumina® sequencing. Examples of such capture probes and uses thereof are described in U.S. Patent Publication Nos. 2014/0378345 and 2015/0376609, the entire contents of each of which are incorporated herein by reference.

The capture probe can include one or more spatial barcodes (e.g., two or more, three or more, four or more, five or more) spatial barcodes. A “spatial barcode” is a contiguous nucleic acid segment or two or more non-contiguous nucleic acid segments that function as a label or identifier that conveys or is capable of conveying spatial information. In some embodiments, a capture probe includes a spatial barcode that possesses a spatial aspect, where the barcode is associated with a particular location within an array or a particular location on a substrate.

A spatial barcode can be part of an analyte, or independent from an analyte (e.g., part of the capture probe). A spatial barcode can be a tag attached to an analyte (e.g., a nucleic acid molecule) or a combination of a tag in addition to an endogenous characteristic of the analyte (e.g., size of the analyte or end sequence(s)). A spatial barcode can be unique. In some embodiments where the spatial barcode is unique, the spatial barcode functions both as a spatial barcode and as a unique molecular identifier (UMI), associated with one particular capture probe.

Spatial barcodes can have a variety of different formats. For example, spatial barcodes can include polynucleotide spatial barcodes; random nucleic acid and/or amino acid sequences; and synthetic nucleic acid and/or amino acid sequences. In some embodiments, a spatial barcode is attached to an analyte in a reversible or irreversible manner. In some embodiments, a spatial barcode is added to, for example, a fragment of a DNA or RNA sample before, during, and/or after sequencing of the sample. In some embodiments, a spatial barcode allows for identification and/or quantification of individual sequencing-reads. In some embodiments, a spatial barcode is a used as a fluorescent barcode for which fluorescently labeled oligonucleotide probes hybridize to the spatial barcode.

In some embodiments, the spatial barcode is a nucleic acid sequence that does not substantially hybridize to analyte nucleic acid molecules in a biological sample. In some embodiments, the spatial barcode has less than 80% sequence identity (e.g., less than 70%, 60%, 50%, or less than 40% sequence identity) to the nucleic acid sequences across a substantial part (e.g., 80% or more) of the nucleic acid molecules in the biological sample.

The spatial barcode sequences can include from about 6 to about 20 or more nucleotides within the sequence of the capture probes. In some embodiments, the length of a spatial barcode sequence can be about 6, 7, 8, 9, 10, 11, 12, 13, 14, 15, 16, 17, 18, 19, 20 nucleotides or longer. In some embodiments, the length of a spatial barcode sequence can be at least about 6, 7, 8, 9, 10, 11, 12, 13, 14, 15, 16, 17, 18, 19, 20 nucleotides or longer. In some embodiments, the length of a spatial barcode sequence is at most about 6, 7, 8, 9, 10, 11, 12, 13, 14, 15, 16, 17, 18, 19, 20 nucleotides or shorter.

These nucleotides can be completely contiguous, e.g., in a single stretch of adjacent nucleotides, or they can be separated into two or more separate subsequences that are separated by 1 or more nucleotides. Separated spatial barcode subsequences can be from about 4 to about 16 nucleotides in length. In some embodiments, the spatial barcode subsequence can be about 4, 5, 6, 7, 8, 9, 10, 11, 12, 13, 14, 15, 16 nucleotides or longer. In some embodiments, the spatial barcode subsequence can be at least about 4, 5, 6, 7, 8, 9, 10, 11, 12, 13, 14, 15, 16 nucleotides or longer. In some embodiments, the spatial barcode subsequence can be at most about 4, 5, 6, 7, 8, 9, 10, 11, 12, 13, 14, 15, 16 nucleotides or shorter.

For multiple capture probes that are attached to a common array feature, the one or more spatial barcode sequences of the multiple capture probes can include sequences that are the same for all capture probes coupled to the feature, and/or sequences that are different across all capture probes coupled to the feature.

The feature can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode. One type of capture probe associated with the feature includes the spatial barcode in combination with a poly(T) capture domain, designed to capture mRNA target analytes. A second type of capture probe associated with the feature includes the spatial barcode in combination with a random N-mer capture domain for gDNA analysis. A third type of capture probe associated with the feature includes the spatial barcode in combination with a capture domain complementary to the analyte capture agent of interest. A fourth type of capture probe associated with the feature includes the spatial barcode in combination with a capture probe that can bind a nucleic acid molecule that can function in a CRISPR assay (e.g., CRISPR/Cas9). For example, the schemes can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents.

Capture probes attached to a single array feature can include identical (or common) spatial barcode sequences, different spatial barcode sequences, or a combination of both. Capture probes attached to a feature can include multiple sets of capture probes. Capture probes of a given set can include identical spatial barcode sequences. The identical spatial barcode sequences can be different from spatial barcode sequences of capture probes of another set.

The plurality of capture probes can include spatial barcode sequences (e.g., nucleic acid barcode sequences) that are associated with specific locations on a spatial array. For example, a first plurality of capture probes can be associated with a first region, based on a spatial barcode sequence common to the capture probes within the first region, and a second plurality of capture probes can be associated with a second region, based on a spatial barcode sequence common to the capture probes within the second region. The second region may or may not be associated with the first region. Additional pluralities of capture probes can be associated with spatial barcode sequences common to the capture probes within other regions. In some embodiments, the spatial barcode sequences can be the same across a plurality of capture probe molecules.

In some embodiments, multiple different spatial barcodes are incorporated into a single arrayed capture probe. For example, a mixed but known set of spatial barcode sequences can provide a stronger address or attribution of the spatial barcodes to a given spot or location, by providing duplicate or independent confirmation of the identity of the location. In some embodiments, the multiple spatial barcodes represent increasing specificity of the location of the particular array point.

The capture probe can include one or more (e.g., two or more, three or more, four or more, five or more) Unique Molecular Identifiers (UMIs). A UMI is a contiguous nucleic acid segment or two or more non-contiguous nucleic acid segments that function as a label or identifier for a particular analyte, or for a capture probe that binds a particular analyte (e.g., via the capture domain).

A UMI can be unique. A UMI can include one or more specific polynucleotide sequences, one or more random nucleic acid and/or amino acid sequences, and/or one or more synthetic nucleic acid and/or amino acid sequences, or combinations thereof.

In some embodiments, the UMI is a nucleic acid sequence that does not substantially hybridize to analyte nucleic acid molecules in a biological sample. In some embodiments, the UMI has less than 80% sequence identity (e.g., less than 70%, 60%, 50%, or less than 40% sequence identity) to the nucleic acid sequences across a substantial part (e.g., 80% or more) of the nucleic acid molecules in the biological sample.

The UMI can include from about 6 to about 20 or more nucleotides within the sequence of the capture probes. In some embodiments, the length of a UMI sequence can be about 6, 7, 8, 9, 10, 11, 12, 13, 14, 15, 16, 17, 18, 19, 20 nucleotides or longer. In some embodiments, the length of a UMI sequence can be at least about 6, 7, 8, 9, 10, 11, 12, 13, 14, 15, 16, 17, 18, 19, 20 nucleotides or longer. In some embodiments, the length of a UMI sequence is at most about 6, 7, 8, 9, 10, 11, 12, 13, 14, 15, 16, 17, 18, 19, 20 nucleotides or shorter.

These nucleotides can be completely contiguous, i.e., in a single stretch of adjacent nucleotides, or they can be separated into two or more separate subsequences that are separated by 1 or more nucleotides. Separated UMI subsequences can be from about 4 to about 16 nucleotides in length. In some embodiments, the UMI subsequence can be about 4, 5, 6, 7, 8, 9, 10, 11, 12, 13, 14, 15, 16 nucleotides or longer. In some embodiments, the UMI subsequence can be at least about 4, 5, 6, 7, 8, 9, 10, 11, 12, 13, 14, 15, 16 nucleotides or longer. In some embodiments, the UMI subsequence can be at most about 4, 5, 6, 7, 8, 9, 10, 11, 12, 13, 14, 15, 16 nucleotides or shorter.

In some embodiments, a UMI is attached to an analyte in a reversible or irreversible manner. In some embodiments, a UMI is added to, for example, a fragment of a DNA or RNA sample before, during, and/or after sequencing of the analyte. In some embodiments, a UMI allows for identification and/or quantification of individual sequencing-reads. In some embodiments, a UMI is a used as a fluorescent barcode for which fluorescently labeled oligonucleotide probes hybridize to the UMI.

For capture probes that are attached to an array feature, an individual array feature can include one or more capture probes. In some embodiments, an individual array feature includes hundreds or thousands of capture probes. In some embodiments, the capture probes are associated with a particular individual feature, where the individual feature contains a capture probe including a spatial barcode unique to a defined region or location on the array.

In some embodiments, a particular feature can contain capture probes including more than one spatial barcode (e.g., one capture probe at a particular feature can include a spatial barcode that is different than the spatial barcode included in another capture probe at the same particular feature, while both capture probes include a second, common spatial barcode), where each spatial barcode corresponds to a particular defined region or location on the array. For example, multiple spatial barcode sequences associated with one particular feature on an array can provide a stronger address or attribution to a given location by providing duplicate or independent confirmation of the location. In some embodiments, the multiple spatial barcodes represent increasing specificity of the location of the particular array point. In a non-limiting example, a particular array point can be coded with two different spatial barcodes, where each spatial barcode identifies a particular defined region within the array, and an array point possessing both spatial barcodes identifies the sub-region where two defined regions overlap, e.g., such as the overlapping portion of a Venn diagram.

In another non-limiting example, a particular array point can be coded with three different spatial barcodes, where the first spatial barcode identifies a first region within the array, the second spatial barcode identifies a second region, where the second region is a subregion entirely within the first region, and the third spatial barcode identifies a third region, where the third region is a subregion entirely within the first and second subregions.

In some embodiments, capture probes can further comprise a cleavage sequence, wherein the cleavage sequence is positioned 5′ to the capture domain, and the capture probes are attached via their 5′ ends to a substrate.

In some embodiments, capture probes attached to array features are released from the array features for sequencing. Alternatively, in some embodiments, capture probes remain attached to the array features, and the probes are sequenced while remaining attached to the array features (e.g., via in situ sequencing). Further aspects of the sequencing of capture probes are described in subsequent sections of this disclosure.

In some embodiments, an array feature can include different types of capture probes attached to the feature. For example, the array feature can include a first type of capture probe with a capture domain designed to bind to one type of analyte, and a second type of capture probe with a capture domain designed to bind to a second type of analyte. In general, array features can include one or more (e.g., two or more, three or more, four or more, five or more, six or more, eight or more, ten or more, 12 or more, 15 or more, 20 or more, 30 or more, 50 or more) different types of capture probes attached to a single array feature.

In some embodiments, the capture probe is a nucleic acid. In some embodiments, the capture probe is attached to the array feature via its 5′ end. In some embodiments, the capture probe includes from the 5′ to 3′ end: one or more barcodes (e.g., a spatial barcode and/or a UMI) and one or more capture domains. In some embodiments, the capture probe includes from the 5′ to 3′ end: one barcode (e.g., a spatial barcode or a UMI) and one capture domain. In some embodiments, the capture probe includes from the 5′ to 3′ end: a cleavage sequence or a photocleavable linker, a functional domain, one or more barcodes (e.g., a spatial barcode and/or a UMI), and a capture domain. In some embodiments, the capture probe includes from the 5′ to 3′ end: a cleavage sequence or a photocleavable linker, a functional domain, one or more barcodes (e.g., a spatial barcode), a UMI, a second functional domain, and a capture domain. In some embodiments, the capture probe includes from the 5′ to 3′ end: a cleavage sequence or a photocleavable linker, a functional domain, a spatial barcode, a UMI, and a capture domain. In some embodiments, the capture probe does not include a spatial barcode. In some embodiments, the capture probe does not include a UMI. In some embodiments, the capture probe includes a sequence for initiating a sequencing reaction. In some embodiments, the capture probe includes from the 5′ to 3′ end: a cleavage sequence, a spatial barcode, and a capture domain. In some embodiments, the capture probe includes from the 5′ to 3′ end: a spatial barcode, a cleavage sequence, and a capture domain. In some embodiments, the capture probe includes from the 5′ to 3′ end: a first cleavage sequence, a spatial barcode, a second cleavage sequence, and a capture domain.

In some embodiments, the capture probe is immobilized on a feature via its 3′ end. In some embodiments, the capture probe includes from the 3′ to 5′ end: one or more barcodes (e.g., a spatial barcode), a UMI, and one or more capture domains. In some embodiments, the capture probe includes from the 3′ to 5′ end: one barcode (e.g., a spatial barcode), a UMI, and one capture domain. In some embodiments, the capture probe includes from the 3′ to 5′ end: a photocleavable linker or a cleavage sequence, a functional domain, one or more barcodes (e.g., a spatial barcode), a UMI and a capture domain. In some embodiments, the capture probe includes from the 3′ to 5′ end: a cleavage photocleavable linker or a cleavage sequence, a functional domain, a spatial barcode, a UMI, and a capture domain.

In some embodiments, a capture probe includes an in situ synthesized oligonucleotide. The in situ synthesized oligonucleotide can be attached to a substrate, or to a feature on a substrate. In some embodiments, the in situ synthesized oligonucleotide includes one or more constant sequences, one or more of which serves as a priming sequence (e.g., a primer for amplifying target nucleic acids). The in situ synthesized oligonucleotide can, for example, include a constant sequence at the 3′end that is attached to a substrate, or attached to a feature on a substrate. Additionally or alternatively, the in situ synthesized oligonucleotide can include a constant sequence at the free 5′ end. In some embodiments, the one or more constant sequences can be a cleavable sequence. In some embodiments, the in situ synthesized oligonucleotide includes a barcode sequence, e.g., a variable barcode sequence. The barcode can be any of the barcodes described herein. The length of the barcode can be approximately 8 to 16 nucleotides (e.g., 8, 9, 10, 11, 12, 13, 14, 15, or 16 nucleotides). The length of the in situ synthesized oligonucleotide can be less than 100 nucleotides (e.g., less than 90, 80, 75, 70, 60, 50, 45, 40, 35, 30, 25 or 20 nucleotides). In some instances, the length of the in situ synthesized oligonucleotide is about 20 to about 40 nucleotides. Exemplary in situ synthesized oligonucleotides are produced by Affymetrix. In some embodiments, the in situ synthesized oligonucleotide is attached to a feature of an array.

Additional oligonucleotides can be ligated to an in situ synthesized oligonucleotide to generate a capture probe. For example, a primer complementary to a portion of the in situ synthesized oligonucleotide (e.g., a constant sequence in the oligonucleotide) can be used to hybridize an additional oligonucleotide and extend (using the in situ synthesized oligonucleotide as a template e.g., a primer extension reaction) to form a double stranded oligonucleotide and to further create a 3′ overhang. In some embodiments, the 3′ overhang can be created by template-independent ligases (e.g., terminal deoxynucleotidyl transferase (TdT) or poly(A) polymerase). An additional oligonucleotide comprising one or more capture domains can be ligated to the 3′ overhang using a suitable enzyme (e.g., a ligase), in some embodiments further in combination with a splint oligonucleotide, to generate a capture probe. Thus, in some embodiments, a capture probe is a product of two or more oligonucleotide sequences, (e.g., the in situ synthesized oligonucleotide and the additional oligonucleotide) that are ligated together. In some embodiments, one of the oligonucleotide sequences is an in situ synthesized oligonucleotide.

In some embodiments, the capture probe can be prepared using a splint oligonucleotide. Two or more oligonucleotides can be ligated together using a splint oligonucleotide and any variety of ligases known in the art or described herein (e.g., SplintR ligase).

One of the oligonucleotides can include, for example, a constant sequence (e.g., a sequence complementary to a portion of a splint oligonucleotide), a degenerate sequence, and/or a capture domain (e.g., as described herein). One of the oligonucleotides can also include a sequence compatible for ligating or hybridizing to an analyte of interest in the biological sample. An analyte of interest (e.g., an mRNA) can also be used as a splint oligonucleotide to facilitate the ligation of additional oligonucleotides onto the capture probe. In some embodiments, the capture probe is generated by having an enzyme add polynucleotides at the end of an oligonucleotide sequence. The capture probe can include a degenerate sequence, which can function as a unique molecular identifier.

A degenerate sequence can be a degenerate nucleotide sequence including about or at least 5, 6, 7, 8, 9, 10, 11, 12, 13, 14, 15, 16, 17, 18, 19, 20, 25, 30, 35, 40, 45, or 50 nucleotides. In some embodiments, a nucleotide sequence contains 1, 2, 3, 4, 5, 6, 7, 8, 9, 10, 15, 20, 25, or more degenerate positions within the nucleotide sequence. In some embodiments, the degenerate sequence is used as a UMI.

In some embodiments, the hybridization of a cleavage probe to a cleavage sequence in a capture probe results in the formulation of a restriction endonuclease recognition sequence or a sequence of nucleotides cleavable by specific enzyme activities. In some embodiments, a cleavage domain in a capture probe can comprise a sequence of nucleotides cleavable by specific enzyme activities. For example, uracil sequences can be enzymatically cleaved from a nucleotide sequence using uracil DNA glycosylase (UDG) or Uracil Specific Excision Reagent (USER). As another example, other modified bases (e.g., modified by methylation) can be recognized and cleaved by specific endonucleases.

In some embodiments, where a blocking domain is used, the capture probes can be subjected to an enzymatic cleavage, which removes the blocking domain and any of the additional nucleotides that are added to the 3′ end of the capture probe during the modification process. Removal of the blocking domain reveals and/or restores the free 3′ end of the capture domain of the capture probe. In some embodiments, additional nucleotides can be removed to reveal and/or restore the 3′ end of the capture domain of the capture probe.

In some embodiments, a blocking domain can be incorporated into the capture probe when it is synthesized, or after its synthesis. The terminal nucleotide of the capture domain is a reversible terminator nucleotide (e.g., 3′-O-blocked reversible terminator and 3′-unblocked reversible terminator), and can be included in the capture probe during or after probe synthesis.

This disclosure also provides methods and materials for using analyte capture agents for spatial profiling of biological analytes (e.g., mRNA, genomic DNA, accessible chromatin, and cell surface or intracellular proteins and/or metabolites). As used herein, an “analyte capture agent” refers to an agent that interacts with an analyte (e.g., an analyte in a sample) and with a capture probe (e.g., a capture probe attached to a substrate) to identify the analyte. In some embodiments, the analyte capture agent includes an analyte binding moiety and a capture agent barcode domain.

An analyte binding moiety is a molecule capable of binding to an analyte and interacting with a spatially-barcoded capture probe. The analyte binding moiety can bind to the analyte with high affinity and/or with high specificity. The analyte capture agent can include a capture agent barcode domain, a nucleotide sequence (e.g., an oligonucleotide), which can hybridize to at least a portion or an entirety of a capture domain of a capture probe. The analyte binding moiety can include a polypeptide and/or an aptamer (e.g., an oligonucleotide or peptide molecule that binds to a specific target analyte). The analyte binding moiety can include an antibody or antibody fragment (e.g., an antigen-binding fragment).

As used herein, the term “analyte binding moiety” refers to a molecule or moiety capable of binding to a macromolecular constituent (e.g., an analyte, e.g., a biological analyte). In some embodiments of any of the spatial profiling methods described herein, the analyte binding moiety of the analyte capture agent that binds to a biological analyte can include, but is not limited to, an antibody, or an epitope binding fragment thereof, a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The analyte binding moiety can bind to the macromolecular constituent (e.g., analyte) with high affinity and/or with high specificity. The analyte binding moiety can include a nucleotide sequence (e.g., an oligonucleotide), which can correspond to at least a portion or an entirety of the analyte binding moiety. The analyte binding moiety can include a polypeptide and/or an aptamer (e.g., a polypeptide and/or an aptamer that binds to a specific target molecule, e.g., an analyte). The analyte binding moiety can include an antibody or antibody fragment (e.g., an antigen-binding fragment) that binds to a specific analyte (e.g., a polypeptide).

In some embodiments, an analyte binding moiety of an analyte capture agent includes one or more antibodies or antigen binding fragments thereof. The antibodies or antigen binding fragments including the analyte binding moiety can bind to a target analyte. In some embodiments, the analyte is a protein (e.g., a protein on a surface of the biological sample (e.g., a cell) or an intracellular protein). In some embodiments, a plurality of analyte capture agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte capture agents are the same. In some embodiments in which the plurality of analytes includes a single species of analyte, the analyte binding moieties of the plurality of analyte capture agents are the different (e.g., members of the plurality of analyte capture agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

An analyte capture agent can include an analyte binding moiety. The analyte binding moiety can be an antibody. Exemplary, non-limiting antibodies that can be used as analyte binding moieties in an analyte capture agent or that can be used in the IHC/IF applications disclosed herein include any of the following including variations thereof: A-ACT, A-AT, ACTH, Actin-Muscle-specific, Actin-Smooth Muscle (SMA), AE1, AE1/AE3, AE3, AFP, AKT Phosphate, ALK-1, Amyloid A, Androgen Receptor, Annexin Al, B72.3, BCA-225, BCL-1 (Cyclin D1), BCL-1/CD20, BCL-2, BCL-2/BCL-6, BCL-6, Ber-EP4, Beta-amyloid, Beta-catenin, BG8 (Lewis Y), BOB-1, CA 19.9, CA 125, CAIX, Calcitonin, Caldesmon, Calponin, Calretinin, CAM 5.2, CAM 5.2/AE1, CDla, CD2, CD3 (M), CD3 (P), CD3/CD20, CD4, CDS, CD7, CD8, CD10, CD14, CD15, CD20, CD21, CD22, CD 23, CD25, CD30, CD31, CD33, CD34, CD35, CD43, CD45 (LCA), CD45RA, CD56, CD57, CD61, CD68, CD71, CD74, CD79a, CD99, CD117 (c-KIT), CD123, CD138, CD163, CDX-2, CDX-2/CK-7, CEA (M), CEA (P), Chromogranin A, Chymotrypsin, CK-5, CK-5/6, CK-7, CK-7/TTF-1, CK-14, CK-17, CK-18, CK-19, CK-20, CK-HMW, CK-LMW, CMV-IH, COLL-IV, COX-2, D2-40, DBA44, Desmin, DOG1, EBER-ISH, EBV (LMP1), E-Cadherin, EGFR, EMA, ER, ERCC1, Factor VIII (vWF), Factor XIIIa, Fascin, FLI-1, FHS, Galectin-3, Gastrin, GCDFP-15, GFAP, Glucagon, Glycophorin A, Glypican-3, Granzyme B, Growth Hormone (GH), GST, HAM 56, HMBE-1, HBP, HCAg, HCG, Hemoglobin A, HEP B CORE (HBcAg), HEP B SURF, (HBsAg), HepPar1, HER2, Herpes I, Herpes II, HHV-8, HLA-DR, HMB 45, HPL, HPV-IHC, HPV (6/11)-ISH, HPV (16/18)-ISH, HPV (31/33)-ISH, HPV WSS-ISH, HPV High-ISH, HPV Low-ISH, HPV High & Low-ISH, IgA, IgD, IgG, IgG4, IgM, Inhibin, Insulin, JC Virus-ISH, Kappa-ISH, KER PAN, Ki-67, Lambda-IHC, Lambda-ISH, LH, Lipase, Lysozyme (MURA), Mammaglobin, MART-1, MBP, M-Cell Tryptase, MEL-5, Melan-A, Melan-A/Ki-67, Mesothelin, MiTF, MLH-1, MOC-31, MPO, MSH-2, MSH-6, MUC1, MUC2, MUC4, MUCSAC, MUM-1, MVO D1, Myogenin, Myoglobin, Myoin Heavy Chain, Napsin A, NB84a, NEW-N, NF, NK1-C3, NPM, NSE, OCT-2, OCT-3/4, OSCAR, p16, p21, p27/Kip1, p53, p57, p63, p120, P504S, Pan Melanoma, PANC.POLY, Parvovirus B19, PAX-2, PAX-5, PAX-5/CD43, PAX=5/CDS, PAX-8, PC, PD1, Perforin, PGP 9.5, PLAP, PMS-2, PR, Prolactin, PSA, PSAP, PSMA, PTEN, PTH, PTS, RB, RCC, S6, S100, Serotonin, Somatostatin, Surfactant (SP-A), Synaptophysin, Synuclein, TAU, TCL-1, TCR beta, TdT, Thrombomodulin, Thyroglobulin, TIA-1, TOXO, TRAP, TriView™ breast, TriView™ prostate, Trypsin, TS, TSH, TTF-1, Tyrosinase, Ubiqutin, Uroplakin, VEGF, Villin, Vimentin (VIM), VIP, VZV, WT1 (M) N-Terminus, WTI (P) C-Terminus, ZAP-70.

Further, exemplary, non-limiting antibodies that can be used as analyte binding moieties in an analyte capture agent or that can be used in the IHC/IF applications disclosed herein include any of the following antibodies (and variations thereof) to: cell surface proteins, intracellular proteins, kinases (e.g., AGC kinase family (e.g., AKT1, AKT2, PDK1, Protein Kinase C, ROCK1, ROCK2, SGK3), CAMK kinase family (e.g., AMPK1, AMPK2, CAMK, Chk1, Chk2, Zip), CK1 kinase family, TK kinase family (e.g., Abl2, AXL, CD167, CD246/ALK, c-Met, CSK, c-Src, EGFR, ErbB2 (HER2/neu), ErbB3, ErbB4, FAK, Fyn, LCK, Lyn, PKT7, Syk, Zap70), STE kinase family (e.g., ASK1, MAPK, MEK1, MEK2, MEK3 MEK4, MEKS, PAK1, PAK2, PAK4, PAK6), CMGC kinase family (e.g., Cdk2, Cdk4, Cdk5, Cdk6, Cdk7, Cdk9, Erk1, GSK3, Jnk/MAPK8, Jnk2/MAPK9, JNK3/MAPK10, p38/MAPK), and TKL kinase family (e.g., ALK1, ILK1, IRAK1, IRAK2, IRAK3, IRAK4, LIMK1, LIMK2, M3K11, RAF1, RIP1, RIP3, VEGFR1, VEGFR2, VEGFR3), Aurora A kinase, Aurora B kinase, IKK, Nemo-like kinase, PINK, PLK3, ULK2, WEE1, transcription factors (e.g., FOXP3, ATF3, BACH1, EGR, ELF3, FOXA1, FOXA2, FOX01, GATA), growth factor receptors, tumor suppressors (e.g., anti-p53, anti-BLM, anti-Cdk2, anti-Chk2, anti-BRCA-1, anti-NBS1, anti-BRCA-2, anti-WRN, anti-PTEN, anti-WT1, anti-p38).

In some embodiments, analyte capture agents are capable of binding to analytes present inside a cell. In some embodiments, analyte capture agents are capable of binding to cell surface analytes that can include, without limitation, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction. In some embodiments, the analyte capture agents are capable of binding to cell surface analytes that are post-translationally modified. In such embodiments, analyte capture agents can be specific for cell surface analytes based on a given state of posttranslational modification (e.g., phosphorylation, glycosylation, ubiquitination, nitrosylation, methylation, acetylation or lipidation), such that a cell surface analyte profile can include posttranslational modification information of one or more analytes.

In some embodiments, the analyte capture agent includes a capture agent barcode domain that is conjugated or otherwise attached to the analyte binding moiety. In some embodiments, the capture agent barcode domain is covalently-linked to the analyte binding moiety. In some embodiments, the capture agent barcode domain is reversibly linked to the analyte binding moiety. In some embodiments, a capture agent barcode domain is a nucleic acid sequence. In some embodiments, a capture agent barcode domain includes an analyte binding moiety barcode and an analyte capture sequence.

As used herein, the term “analyte binding moiety barcode” refers to a barcode that is associated with or otherwise identifies the analyte binding moiety. In some embodiments, by identifying an analyte binding moiety and its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein. For example, an analyte capture agent that is specific to one type of analyte can have coupled thereto a first capture agent barcode domain (e.g., that includes a first analyte binding moiety barcode), while an analyte capture agent that is specific to a different analyte can have a different capture agent barcode domain (e.g., that includes a second barcode analyte binding moiety barcode) coupled thereto. In some aspects, such a capture agent barcode domain can include an analyte binding moiety barcode that permits identification of the analyte binding moiety to which the capture agent barcode domain is coupled. The selection of the capture agent barcode domain can allow significant diversity in terms of sequence, while also being readily attachable to most analyte binding moieties (e.g., antibodies or aptamers) as well as being readily detected, (e.g., using sequencing or array technologies).

In some embodiments, the capture agent barcode domain of an analyte capture agent includes an analyte capture sequence. As used herein, the term “analyte capture sequence” refers to a region or moiety configured to hybridize to a capture domain of a capture probe. In some embodiments, an analyte capture sequence includes a nucleic acid sequence that is complementary to or substantially complementary to the capture domain of a capture probe such that the analyte capture sequence hybridizes to the capture domain of the capture probe. In some embodiments, an analyte capture sequence comprises a poly(A) nucleic acid sequence that hybridizes to a capture domain that comprises a poly(T) nucleic acid sequence. In some embodiments, an analyte capture sequence comprises a poly(T) nucleic acid sequence that hybridizes to a capture domain that comprises a poly(A) nucleic acid sequence. In some embodiments, an analyte capture sequence comprises a non-homopolymeric nucleic acid sequence that hybridizes to a capture domain that comprises a non-homopolymeric nucleic acid sequence that is complementary (or substantially complementary) to the non-homopolymeric nucleic acid sequence of the analyte capture region.

In some embodiments of any of the spatial analysis methods described herein that employ an analyte capture agent, the capture agent barcode domain can be directly coupled to the analyte binding moiety, or they can be attached to a bead, molecular lattice, e.g., a linear, globular, cross-slinked, or other polymer, or other framework that is attached or otherwise associated with the analyte binding moiety, which allows attachment of multiple capture agent barcode domains to a single analyte binding moiety. Attachment (coupling) of the capture agent barcode domains to the analyte binding moieties can be achieved through any of a variety of direct or indirect, covalent or non-covalent associations or attachments. For example, in the case of a capture agent barcode domain coupled to an analyte binding moiety that includes an antibody or antigen-binding fragment, such capture agent barcode domains can be covalently attached to a portion of the antibody or antigen-binding fragment using chemical conjugation techniques (e.g., Lightning-Link® antibody labelling kits available from Innova Biosciences). In some embodiments, a capture agent barcode domain can be coupled to an antibody or antigen-binding fragment using non-covalent attachment mechanisms (e.g., using biotinylated antibodies and oligonucleotides or beads that include one or more biotinylated linker(s), coupled to oligonucleotides with an avidin or streptavidin linker.) Antibody and oligonucleotide biotinylation techniques can be used, and are described for example in Fang et al., Nucleic Acids Res. (2003), 31(2): 708-715, the entire contents of which are incorporated by reference herein. Likewise, protein and peptide biotinylation techniques have been developed and can be used, and are described for example in U.S. Pat. No. 6,265,552, the entire contents of which are incorporated by reference herein. Furthermore, click reaction chemistry such as a methyltetrazine-PEGS-NHS ester reaction, a TCO-PEG4-NHS ester reaction, or the like, can be used to couple capture agent barcode domains to analyte binding moieties. The reactive moiety on the analyte binding moiety can also include amine for targeting aldehydes, amine for targeting maleimide (e.g., free thiols), azide for targeting click chemistry compounds (e.g., alkynes), biotin for targeting streptavidin, phosphates for targeting EDC, which in turn targets active ester (e.g., NH2). The reactive moiety on the analyte binding moiety can be a chemical compound or group that binds to the reactive moiety on the analyte binding moiety. Exemplary strategies to conjugate the analyte binding moiety to the capture agent barcode domain include the use of commercial kits (e.g., Solulink, Thunder link), conjugation of mild reduction of hinge region and maleimide labelling, stain-promoted click chemistry reaction to labeled amides (e.g., copper-free), and conjugation of periodate oxidation of sugar chain and amine conjugation. In the cases where the analyte binding moiety is an antibody, the antibody can be modified prior to or contemporaneously with conjugation of the oligonucleotide. For example, the antibody can be glycosylated with a chemical substrate-permissive mutant of β-1,4-galactosyltransferase, GalT (Y289L) and azide-bearing uridine diphosphate-N-acetylgalactosamine analog uridine diphosphate -GalNAz. The modified antibody can be conjugated to an oligonucleotide with a dibenzocyclooctyne-PEG4-NHS group. In some embodiments, certain steps (e.g., COOH activation (e.g., EDC) and homobifunctional cross linkers) can be avoided to prevent the analyte binding moieties from conjugating to themselves. In some embodiments of any of the spatial profiling methods described herein, the analyte capture agent (e.g., analyte binding moiety coupled to an oligonucleotide) can be delivered into the cell, e.g., by transfection (e.g., using transfectamine, cationic polymers, calcium phosphate or electroporation), by transduction (e.g., using a bacteriophage or recombinant viral vector), by mechanical delivery (e.g., magnetic beads), by lipid (e.g., 1,2-Dioleoyl-sn-glycero-3-phosphocholine (DOPC)), or by transporter proteins. An analyte capture agent can be delivered into a cell using exosomes. For example, a first cell can be generated that releases exosomes comprising an analyte capture agent. An analyte capture agent can be attached to an exosome membrane. An analyte capture agent can be contained within the cytosol of an exosome. Released exosomes can be harvested and provided to a second cell, thereby delivering the analyte capture agent into the second cell. An analyte capture agent can be releasable from an exosome membrane before, during, or after delivery into a cell. In some embodiments, the cell is permeabilized to allow the analyte capture agent to couple with intracellular constituents (such as, without limitation, intracellular proteins, metabolites, and nuclear membrane proteins). Following intracellular delivery, analyte capture agents can be used to analyze intracellular constituents as described herein.

In some embodiments of any of the spatial profiling methods described herein, the capture agent barcode domain coupled to an analyte capture agent can include modifications that render it non-extendable by a polymerase. In some embodiments, when binding to a capture domain of a capture probe or nucleic acid in a sample for a primer extension reaction, the capture agent barcode domain can serve as a template, not a primer. When the capture agent barcode domain also includes a barcode (e.g., an analyte binding moiety barcode), such a design can increase the efficiency of molecular barcoding by increasing the affinity between the capture agent barcode domain and unbarcoded sample nucleic acids, and eliminate the potential formation of adaptor artifacts. In some embodiments, the capture agent barcode domain can include a random N-mer sequence that is capped with modifications that render it non-extendable by a polymerase. In some cases, the composition of the random N-mer sequence can be designed to maximize the binding efficiency to free, unbarcoded ssDNA molecules. The design can include a random sequence composition with a higher GC content, a partial random sequence with fixed G or C at specific positions, the use of guanosines, the use of locked nucleic acids, or any combination thereof.

A modification for blocking primer extension by a polymerase can be a carbon spacer group of different lengths or a dideoxynucleotide. In some embodiments, the modification can be an abasic site that has an apurine or apyrimidine structure, a base analog, or an analogue of a phosphate backbone, such as a backbone of N-(2-aminoethyl)-glycine linked by amide bonds, tetrahydrofuran, or 1′, 2′-Dideoxyribose. The modification can also be a uracil base, 2′OMe modified RNA, C3-18 spacers (e.g., structures with 3-18 consecutive carbon atoms, such as C3 spacer), ethylene glycol multimer spacers (e.g., spacer 18 (hexa-ethyleneglycol spacer), biotin, di-deoxynucleotide triphosphate, ethylene glycol, amine, or phosphate.

In some embodiments of any of the spatial profiling methods described herein, the capture agent barcode domain coupled to the analyte binding moiety includes a cleavable domain. For example, after the analyte capture agent binds to an analyte (e.g., a cell surface analyte), the capture agent barcode domain can be cleaved and collected for downstream analysis according to the methods as described herein. In some embodiments, the cleavable domain of the capture agent barcode domain includes a U-excising element that allows the species to release from the bead. In some embodiments, the U-excising element can include a single-stranded DNA (ssDNA) sequence that contains at least one uracil. The species can be attached to a bead via the ssDNA sequence. The species can be released by a combination of uracil-DNA glycosylase (e.g., to remove the uracil) and an endonuclease (e.g., to induce an ssDNA break). If the endonuclease generates a 5′ phosphate group from the cleavage, then additional enzyme treatment can be included in downstream processing to eliminate the phosphate group, e.g., prior to ligation of additional sequencing handle elements, e.g., Illumina full P5 sequence, partial P5 sequence, full R1 sequence, and/or partial R1 sequence.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (i.e., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte capture agent comprising an analyte binding moiety that binds to the cell surface protein and an analyte binding moiety barcode that identifies that analyte binding moiety, and the cell can be subjected to spatial analysis (e.g., any of the variety of spatial analysis methods described herein). For example, the analyte capture agent bound to the cell surface protein can be bound to a capture probe (e.g., a capture probe on an array), which capture probe includes a capture domain that interacts with an analyte capture sequence present on the capture agent barcode domain of the analyte capture agent. All or part of the capture agent barcode domain (including the analyte binding moiety barcode) can be copied with a polymerase using a 3′ end of the capture domain as a priming site, generating an extended capture probe that includes the all or part of complementary sequence that corresponds to the capture probe (including a spatial barcode present on the capture probe) and a copy of the analyte binding moiety barcode. In some embodiments, an analyte capture agent with an extended capture agent barcode domain that includes a sequence complementary to a spatial barcode of a capture probe is called a “spatially-tagged analyte capture agent.”

In some embodiments, the spatial array with spatially-tagged analyte capture agents can be contacted with a sample, where the analyte capture agent(s) associated with the spatial array capture the target analyte(s). The analyte capture agent(s) containing the extended capture probe(s), which includes a sequence complementary to the spatial barcode(s) of the capture probe(s) and the analyte binding moiety barcode(s), can then be denatured from the capture probe(s) of the spatial array. This allows the spatial array to be reused. The sample can be dissociated into non-aggregated cells (e.g., single cells) and analyzed by the single cell/droplet methods described herein. The spatially-tagged analyte capture agent can be sequenced to obtain the nucleic acid sequence of the spatial barcode of the capture probe and the analyte binding moiety barcode of the analyte capture agent. The nucleic acid sequence of the extended capture probe can thus be associated with an analyte (e.g., cell surface protein), and in turn, with the one or more physical properties of the cell (e.g., a shape or cell type). In some embodiments, the nucleic acid sequence of the extended capture probe can be associated with an intracellular analyte of a nearby cell, where the intracellular analyte was released using any of the cell permeabilization or analyte migration techniques described herein.

In some embodiments of any of the spatial profiling methods described herein, the capture agent barcode domains released from the analyte capture agents can then be subjected to sequence analysis to identify which analyte capture agents were bound to analytes. Based upon the capture agent barcode domains that are associated with a feature (e.g., a feature at a particular location) on a spatial array and the presence of the analyte binding moiety barcode sequence, an analyte profile can be created for a biological sample. Profiles of individual cells or populations of cells can be compared to profiles from other cells, e.g., ‘normal’ cells, to identify variations in analytes, which can provide diagnostically relevant information. In some embodiments, these profiles can be useful in the diagnosis of a variety of disorders that are characterized by variations in cell surface receptors, such as cancer and other disorders.

In some embodiments, the feature is a bead. In some embodiments, the feature is a gel bead. A “bead” can be a particle. A bead can be porous, non-porous, solid, semi-solid, and/or a combination thereof. In some embodiments, a bead can be dissolvable, disruptable, and/or degradable, whereas in certain embodiments, a bead is not degradable. A semi-solid bead can be a liposomal bead. Solid beads can include metals including, without limitation, iron oxide, gold, and silver. In some embodiments, the bead can be a silica bead. In some embodiments, the bead can be rigid. In some embodiments, the bead can be flexible and/or compressible.

The bead can be a macromolecule. The bead can be formed of nucleic acid molecules bound together. The bead can be formed via covalent or non-covalent assembly of molecules (e.g., macromolecules), such as monomers or polymers. Polymers or monomers can be natural or synthetic. Polymers or monomers can be or include, for example, nucleic acid molecules (e.g., DNA or RNA).

A bead can be rigid, or flexible and/or compressible. A bead can include a coating including one or more polymers. Such a coating can be disruptable or dissolvable. In some embodiments, a bead includes a spectral or optical label (e.g., dye) attached directly or indirectly (e.g., through a linker) to the bead. For example, a bead can be prepared as a colored preparation (e.g., a bead exhibiting a distinct color within the visible spectrum) that can change color (e.g., colorimetric beads) upon application of a desired stimulus (e.g., heat and/or chemical reaction) to form differently colored beads (e.g., opaque and/or clear beads).

A bead can include natural and/or synthetic materials. For example, a bead can include a natural polymer, a synthetic polymer or both natural and synthetic polymers. Examples of natural polymers include, without limitation, proteins, sugars such as deoxyribonucleic acid, rubber, cellulose, starch (e.g., amylose, amylopectin), enzymes, polysaccharides, silks, polyhydroxyalkanoates, chitosan, dextran, collagen, carrageenan, ispaghula, acacia, agar, gelatin, shellac, sterculia gum, xanthan gum, corn sugar gum, guar gum, gum karaya, agarose, alginic acid, alginate, or natural polymers thereof. Examples of synthetic polymers include, without limitation, acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads can also be formed from materials other than polymers, including for example, lipids, micelles, ceramics, glass-ceramics, material composites, metals, and/or other inorganic materials.

In some embodiments, a bead is a degradable bead. A degradable bead can include one or more species (e.g., disulfide linkers, primers, other oligonucleotides, etc.) with a labile bond such that, when the bead/species is exposed to the appropriate stimuli, the labile bond is broken and the bead degrades. The labile bond can be a chemical bond (e.g., covalent bond, ionic bond) or can be another type of physical interaction (e.g., van der Waals interactions, dipole-dipole interactions, etc.). In some embodiments, a cross-linker used to generate a bead can include a labile bond. Upon exposure to the appropriate conditions, the labile bond can be broken and the bead degraded. For example, upon exposure of a polyacrylamide gel bead including cystamine cross-linkers to a reducing agent, the disulfide bonds of the cystamine can be broken and the bead degraded.

Degradation can refer to the disassociation of a bound or entrained species (e.g., disulfide linkers, primers, other oligonucleotides, etc.) from a bead, both with and without structurally degrading the physical bead itself. For example, entrained species can be released from beads through osmotic pressure differences due to, for example, changing chemical environments. By way of example, alteration of bead pore volumes due to osmotic pressure differences can generally occur without structural degradation of the bead itself. In some embodiments, an increase in pore volume due to osmotic swelling of a bead can permit the release of entrained species within the bead. In some embodiments, osmotic shrinking of a bead can cause a bead to better retain an entrained species due to pore volume contraction.

Any suitable agent that can degrade beads can be used. In some embodiments, changes in temperature or pH can be used to degrade thermo-sensitive or pH-sensitive bonds within beads. In some embodiments, chemical degrading agents can be used to degrade chemical bonds within beads by oxidation, reduction or other chemical changes. For example, a chemical degrading agent can be a reducing agent, such as DTT, where DTT can degrade the disulfide bonds formed between a cross-linker and gel precursors, thus degrading the bead. In some embodiments, a reducing agent can be added to degrade the bead, which can cause the bead to release its contents. Examples of reducing agents can include, without limitation, dithiothreitol (DTT), β-mercaptoethanol, (2S)-2-amino-1,4-dimercaptobutane (dithiobutylamine or DTBA), tris(2-carboxyethyl) phosphine (TCEP), or combinations thereof.

In order to increase efficiency, sensitivity, and/or decrease the non-specific binding of analytes to capture domains of capture probes on an area of an array outside of the biological sample (e.g., not covered by the biological sample), provided herein are methods of cleaving capture probes located on a portion of an array not covered by the biological sample.

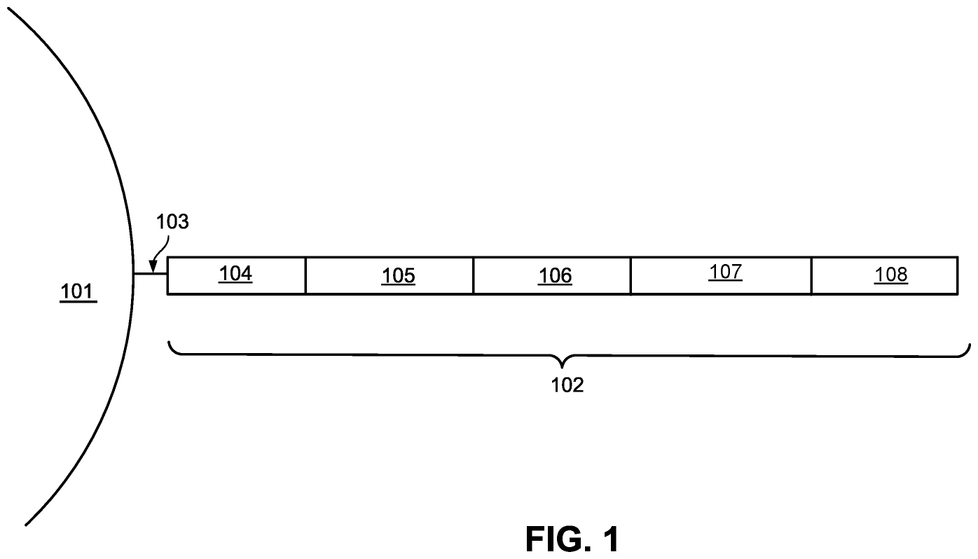

FIG. 1 is a schematic diagram showing an example of a capture probe, as described herein. As shown, the capture probe 102 is optionally coupled to a feature 101 by a cleavable linker 103, such as a photocleavable linker or a chemical cleavage domain. The capture probe can include functional sequences that are useful for subsequent processing, such as functional sequence 104, which can include a sequencer specific flow cell attachment sequence, e.g., a P5 or P7 sequence, as well as functional sequence 105, which can include sequencing primer sequences, e.g., a R1 primer binding site, a R2 primer binding site. In some embodiments, sequence 104 is a P7 sequence and sequence 105 is a R2 primer binding site. A functional sequence 104 or 105 could also be a cleavage sequence. A unique molecular identifier 106 can be included in the capture probe. A spatial barcode 107 can be included within the capture probe for use in barcoding the target analyte. The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 107, functional sequences 104 (e.g., flow cell attachment sequence, cleavage sequence) and 105 (e.g., sequencing primer sequences, cleavage sequence) can be common to all of the probes attached to a given feature. The spatial barcode can also include a capture domain 108 to facilitate capture of a target analyte.

In some embodiments, the photocleavable linker is a photo-sensitive chemical bond (e.g., a chemical bond that dissociates when exposed to light such as ultraviolet light). Cleaving capture probes in an area on the array that is outside of a biological sample (e.g., not covered by the biological sample) can increase the sensitivity and/or efficiency of an assay by preventing analytes and/or probes from being captured on an area of the array outside of where the biological sample is disposed (e.g., not covered by the biological sample). When a photocleavable linker is present, the cleavage reaction is triggered by light, and can be highly selective to the linker and consequently biorthogonal. Typically, wavelength absorption for the photocleavable linker is located in the near-UV range of the spectrum. In some embodiments, λmax of the photocleavable linker is from about 100 nm to about 600 nm, from about 250 nm to about 400 nm, from about 300 nm to about 350 nm, or from about 310 nm to about 365 nm. In some embodiments, λmax of the photocleavable linker is about 100 nm, about 150 nm, about 200 nm, about 225 nm, about 250 nm, about 275 nm, about 300 nm, about 312 nm, about 325 nm, about 330 nm, about 340 nm, about 345 nm, about 355 nm, about 365 nm, about 400 nm, about 450 nm, about 500 nm, about 550 nm, or about 600 nm.

Non-limiting examples of a photo-sensitive chemical bond that can be used in a photocleavable linker include those described in Leriche et al. Bioorg Med Chem. 2012 Jan 15; 20(2):571-82 and U.S. Publication No. 2017/0275669, both of which are incorporated by reference herein in their entireties. For example, photocleavable linkers that comprise photo-sensitive chemical bonds include 3-amino-3-(2-nitrophenyl)propionic acid (ANP), phenacyl ester derivatives, 8-quinolinyl benzenesulfonate, dicoumarin, 6-bromo-7-alkixycoumarin-4-ylmethoxycarbonyl, a bimane-based linker, and a bis-arylhydrazone based linker. In some embodiments, the photo-sensitive bond is part of a photocleavable linker such as an ortho-nitrobenzyl (ONB) linker below:

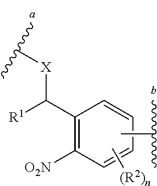

wherein:
X is selected from O and NH;
R¹ is selected from H and C₁₋₃ alkyl;
R² is selected from H and C₁₋₃ alkoxy;
n is 1, 2, or 3; and
a and b each represent either the point of attachment of the photocleavable linker to the substrate, or the point of attachment of the photocleavable linker to the capture probe.

In some embodiments, at least one spacer is included between the substrate and the ortho-nitrobenzyl (ONB) linker, and at least one spacer is included between the ortho-nitrobenzyl (ONB) linker and the capture probe. In some aspects of these embodiments, the spacer comprises at least one group selected from C1-6 alkylene, C2-6 alkenylene, C2-6 alkynylene, C═O, O, S, NH, —(C═O)O—, —(C═O)NH—, —S—S—, ethylene glycol, polyethylene glycol, propylene glycol, and polypropylene glycol, or any combination thereof. In some embodiments, X is O. In some embodiments, X is NH. In some embodiments, R¹ is H. In some embodiments, R¹ is C₁₋₃ alkyl. In some embodiments, R¹ is methyl. In some embodiments, R² is H. In some embodiments, R² is C₁₋₃ alkoxy. In some embodiments, R² is methoxy. In some embodiments, R¹ is H and R² is H. In some embodiments, R¹ is H and R² is methoxy. In some embodiments, R¹ is methyl and R² is H. In some embodiments, R¹ is methyl and R² is methoxy.

In some embodiments, the photocleavable linker has formula:

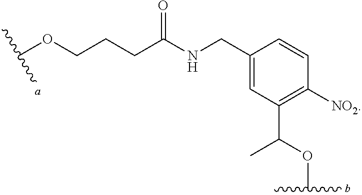 [SMILES: *OCCCC(=O)NCc1ccc([N+](=O)[O-])c(C(C)O*)c1]

In some embodiments, the photocleavable linker has formula:

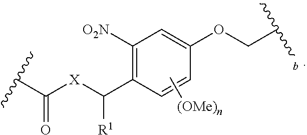

In some embodiments, the photocleavable linker has formula:

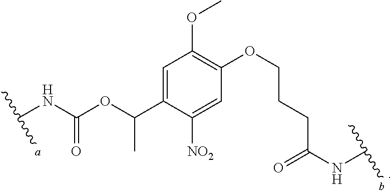 [SMILES: *NC(=O)CCCOc1cc([N+](=O)[O-])c(C(C)OC(=O)N*)cc1OC]

In some embodiments, the photocleavable linker has formula:

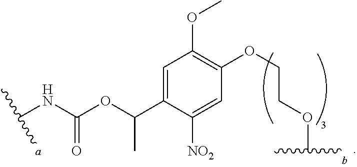

In some embodiments, the photocleavable linker has formula:

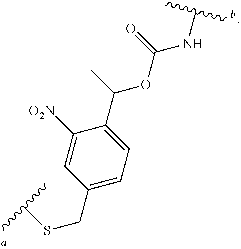 [SMILES: *NC(=O)OC(C)c1ccc(CS*)cc1[N+](=O)[O-]]

Without being bound to any particular theory, it is believed that excitation of the ortho-nitrobenzyl (ONB) linker leads to Norrish-type hydrogen abstraction in the γ-position, followed by formation of azinic acid, which is highly reactive and rearranges into nitroso compound, resulting in the complete cleavage of the linker, as shown on the following scheme:

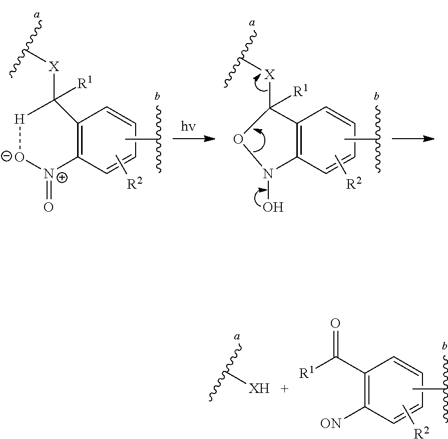

In some embodiments, the photocleavable linker is 3-amino-3-(2-nitrophenyl)propionic acid (ANP) linker:

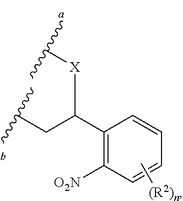

wherein X, R², n, a, and b are as described herein for the ortho-nitrobenzyl (ONB) linker.
   In some embodiments, the photocleavable linker has formula:

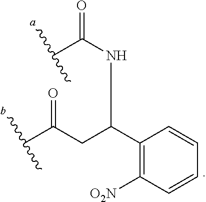 [SMILES: *C(=O)CC(NC(*)=O)c1ccccc1[N+](=O)[O-]]

In some embodiments, the photocleavable linker is phenacyl ester linker:

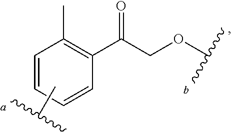

wherein a and b are as described herein for the ortho-nitrobenzyl (ONB) linker.

Other examples of photo-sensitive chemical bonds that can be used in a photocleavable linker include halogenated nucleosides such as bromodeoxyuridine (BrdU). BrdU is an analog of thymidine that can be readily incorporated into oligonucleotides (e.g., in the cleavage domain of a capture probe), and is sensitive to UVB light (280-320 nm range). Upon exposure to UVB light, a photo-cleavage reaction occurs (e.g., at a nucleoside immediately 5′ to the site of BrdU incorporation (Doddridge et al. Chem. Comm., 1998, 18:1997-1998 and Cook et al. Chemistry and Biology. 1999, 6:451-459)) that results in release of the capture probe from the feature.

Non-limiting examples of a photo-sensitive chemical bond that can be used in a photocleavable linker include those described in Leriche et al. Bioorg Med Chem. 2012 Jan 15; 20(2):571-82, U.S. Publication No. 2017/0275669, WO 2020/123305, WO 2020/123311, WO 2020/123309, WO 2020/123317, WO 2020/198071, US 20200277663A1, WO 2020/047007, WO 2020/047004, WO 2020/047002, WO 2020/047005, WO 2020/047010, WO 2020/123318, WO 2020/190509, WO 2020/123301, WO 2020/176788, WO 2020/123320, and WO 2020/123319, each of which are incorporated by reference herein in their entireties.

A photomask can be positioned on the array such that only specific regions of the array are exposed to cleavable stimuli (e.g., exposure to UV light, exposure to light, exposure to heat induced by laser). In some embodiments, the biological sample can be protected by a photomask. In some embodiments, the biological sample can be covered by the photomask to protect the biological sample from light that may damage the biological sample. A photomask can protect the biological sample and the capture probes under the biological sample from exposure to light, thereby protecting a photocleavable linker of the capture probes under the biological sample from cleavage. When the biological sample is protected by a photomask, light can be applied to an array in a non-directed manner. For example, a photomask covering the biological sample can protect the capture probes under the biological from cleaving when light is applied in a non-directed manner to the array.

In some embodiments, methods described herein include exposing the area of the array not covered by the biological sample with a wavelength of light that cleaves the photocleavable linker of the capture probes, thereby releasing the capture probes from the area of the array not covered by the biological sample. In some embodiments, exposing the area of the array not covered by the biological sample includes targeted illumination.

In some embodiments, a light can be directed to an area of the array not covered by the biological sample (e.g., light is directed to capture probes not covered by the biological sample) by a mirror, a mirror array, a lens, or a moving stage. In some embodiments, a scanning laser contacts the area of the array outside biological sample (e.g., scanning laser is directed to capture probes not covered by the biological sample) thereby cleaving the photocleavable linker of the one or more capture probes on the area of the array not covered by the biological sample. In some embodiments, cleaving the photocleavable linker includes heat. In some embodiments, cleaving the photocleavable linker includes UV light.

In some embodiments, the directed light source can include a digital mirror device (DMD) such as a microelectromechanical system (MEMS) digital mirror device (e.g., a digital micro mirror device) which can provide targeted illumination to an area of an array not covered by the biological sample. Non-limiting aspects of DMD methodologies are described in U.S. Pat. No. 5,061,049 and Georgieva et al., arXiv:2010.00955v1, submitted Oct. 2, 2020, and can be used herein in any combination. In some embodiments, the MEMS DMD can provide a light source selected from the group comprising a light emitting diode (LED), laser, Arc, and combinations thereof. In some embodiments, the MEMS DMD can include an imaging device, hardware, and/or software that can detect and image biological samples positioned on an array. In some embodiments, the MEMS DMD can include a microscope. In some embodiments, the MEMS DMD can be operationally compatible with a microscope. In some embodiments, the MEMS DMD can utilize a microscope in conjunction with hardware and/or software to determine the position of a biological sample on an array. In some embodiments, the MEMS DMD can define a region of interest on an array (e.g., can define the boundaries of the biological sample on the array). In some embodiments, after defining the biological sample and/or a region of interest, the MEMS DMD can direct light to the area of the array that is outside of the biological sample (e.g., not covered by the biological sample) or region of interest. In some embodiments, the MEMS DMD can simultaneously define the region of interest and direct light to the area of the array that is outside of the biological sample (e.g., not covered by the biological sample). Non-limiting examples of devices capable of providing directed light sources include ANDOR MOSAIC 3® and LUMINUS® MOSAIC ARRAY SERIES UV CHIP-ON-BOARD LEDS.

Alternatively, enzymatic reactions can be used to cleave capture probes from areas of an array that are not covered by the biological sample. For example, after a biological sample is disposed on an array, an area of the array not covered by the biological sample can be contacted with an enzyme. The enzyme may cleave the capture probes (i.e., capture probes that are in areas of the array not covered by the biological sample) at cleavage sites (i.e., cleavage site specific to the enzyme) and release the capture probes from the area of the array not covered by the biological sample, thus increasing the efficiency, sensitivity, and/or decreasing non-specific binding of analytes. Non-limiting examples of enzymes that are able to cleave a cleavage site in a single-stranded capture probe include AvaII, HaeII, DdeI, AluI, Sau3AI, AccII, TthHB8I, and HapII. In some embodiments, the cleavage sites found in capture probes include a cleavage sequence which hybridizes to a cleavage probe, where upon the enzyme cleaves the cleavage sequence/cleavage probe complex (e.g., cleavage site). The cleavage sites generated by hybridization of cleavage probes to cleavage sequences can be specific to particular enzymes. For example, cleavage sites generated by hybridization of cleavage probes to cleavage sequences can comprise DNA (i.e., cleavage sites for DNase), RNA (i.e., a cleavage sites for RNase), or a combination thereof. For example, hybridization of a cleavage probe to a cleavage sequence can generate a restriction endonuclease cleavage site (e.g., a cleavage site for a restriction enzyme, including, but not limited to EcoRI, EcoRII, BamHI, HindIII, TaqI, NotI, HinFI, Sau3AI, PvuII, SmaI, HaeIII, HgaI, AluI, EcoRV, EcoP15I, KpnI, PstI, SacI, SalI, ScaI, SpeI, SphI, StuI, or XbaI) or a CRISPR/Cas cleavage site (e.g., a cleavage site for a Cas enzyme, such as Cas9, Cas12a (including MAD7), Cas12b, Cas12c, or Cas13). Alternatively, cleavage sites generated by hybridization of cleavage probes to cleavage sequences can comprise cleavage sites for enzymes, such as transposases, integrases, endonucleases, meganucleases, megaTALs, CRISPR-CasX, transcription activator-like effector nucleases (TALEN), or zinc finger nucleases (ZFN). When an array (e.g., areas of an array that are not covered by the biological sample) are exposed to an enzyme that cleaves the cleavage sequence/cleavage probe complex, the enzyme releases the capture probe from the area of the array not covered by the biological sample. Enzymes that can be used in such methods include, without limitation, DNase, RNase, restriction enzymes (e.g., EcoRI, EcoRII, BamHI, HindIII, TaqI, NotI, HinFI, Sau3AI, PvuII, SmaI, HaeIII, HgaI, AluI, EcoRV, EcoP15I, KpnI, PstI, Sad, SalI, ScaI, SpeI, SphI, StuI, XbaI, etc.), Cas enzymes (e.g., Cas9, Cas12a (including MAD7), Cas12b, Cas12c, Cas13, etc.), transposases, integrases, endonucleases, meganucleases, megaTALs, CRISPR-CasX, transcription activator-like effector nucleases (TALENs), or zinc finger nucleases (ZFNs).

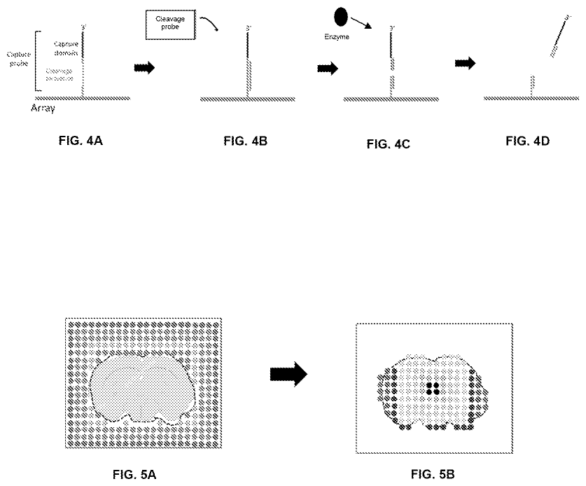

FIGS. 4A-4D provide schematic diagrams showing an exemplary workflow for cleaving capture probes from an area of the array not covered by the biological sample using a cleavage sequence, a cleavage probe and an enzyme. A capture probe for use in the methods described herein can comprise (i) a capture domain, (ii) a cleavage sequence, and (iii) a spatial barcode. FIG. 4A is a schematic diagram showing an example of such a capture probe on the surface of an array. The capture probe can be coupled to one or more features. In some embodiments, the one or more features is a bead (e.g., a gel bead). The capture domain of the capture probe can bind to an analyte of a biological sample. The capture probe can be attached by its 5′ end to the substrate. The capture domain can be positioned 3′ to the cleavage sequence. In some embodiments, the spatial barcode is positioned 5′ to the cleavage sequence. In some embodiments, a cleavage probe can hybridize to the cleavage sequence of a capture probe in an area of the array not covered by the biological sample (FIG. 4B) thereby generating a cleavage site. Unhybridized cleavage probes can optionally be removed from the array (e.g., by washing). When the array (e.g., the area of the array not covered by biological sample) is contacted with an enzyme that cleaves the cleavage sequence hybridized to the cleavage probe, the enzyme cleaves the capture probe (FIG. 4C), thereby releasing the capture probe from the area of the array not covered by the biological sample. The cleaved capture probe can then be removed from the array (e.g., by washing) (FIG. 4D).

An exemplary array for use in the methods described herein can comprise a plurality of subpopulations of capture probes (e.g., capture probes comprising (i) a capture domain, (ii) a cleavage sequence, and (iii) a spatial barcode). Each subpopulation of the plurality of subpopulations of capture probes can comprise capture probes with a common cleavage sequence or a unique cleavage sequence. Each subpopulation of capture probes can be arranged in a geometric pattern on the array. For example, the subpopulations of capture probes can be arranged in a concentric pattern (e.g., a concentric geometric pattern) on the array. FIG. 5A is a schematic diagram showing an example of a tissue section layered on such a patterned array (e.g., an array comprising a plurality of subpopulations of capture probes, wherein the subpopulations of capture probes each have a unique cleavage sequence that differ from other subpopulations and are arranged in a concentric geometric pattern). FIG. 5B is a schematic diagram showing an example of an area of an array covered by the tissue section that has intact capture probes for capturing analytes in the biological sample (e.g., the array shown in FIG. 5A after cleavage of capture probes in areas of the array that are not covered by the tissue section. Capture probes from the same subpopulation of capture probes have the same grey shade in the patterned arrays shown in FIG. 5A and FIG. 5B.

In some embodiments, a biological sample can be disposed (e.g., placed) on an array that includes a plurality of capture probes coupled to a substrate (e.g., a slide). For example, a biological sample can be disposed (e.g., placed) on an array that includes a plurality of capture probes coupled to a substrate (e.g., a slide) via a photocleavable linker. In some embodiments, a biological sample can be disposed (e.g., placed) on an array that includes a plurality of capture probes coupled to a feature (e.g., a bead). For example, a biological sample can be disposed (e.g., placed) on an array that includes a plurality of capture probes coupled to a feature (e.g., a bead) via a photocleavable linker. The methods can further include: exposing the area of the array not covered by the biological sample with a wavelength of light that cleaves the photocleavable linker of the capture probes, thereby releasing the capture probes from the area of the array not covered by the biological sample; removing the cleaved capture probes; contacting an analyte in the biological sample with a capture domain of a capture probe in an area of the array covered by the biological sample; and determining (i) the sequence of the spatial barcode of the capture probe or a complement thereof in the area of the array covered by the biological sample, and (ii) all or a portion of the sequence of the analyte bound to the capture domain of the capture probe or a complement thereof in the area of the array covered by the biological sample, and using the sequences of (i) and (ii) to determine the location of the analyte in the biological sample.

In some embodiments, a biological sample can be disposed (e.g., placed) on an array that includes a plurality of capture probes coupled to a substrate (e.g., a slide), where the plurality of capture probes comprise a capture domain, a cleavage sequence, and a spatial barcode, wherein the cleavage sequence is positioned 5′ to the capture domain, and the capture probes are attached to a substrate via their 5′ end. These methods can further include: contacting an area of the array not covered by the biological sample with a plurality of cleavage probes, wherein a cleavage probe of the plurality of cleavage probes hybridizes to a cleavage sequence in a capture probe in an area of the array not covered by the biological sample; contacting the area of the array not covered by the biological sample with an enzyme that cleaves the cleavage sequence hybridized to the cleavage probe, thereby releasing the capture probe from the area of the array not covered by the biological sample; removing the cleaved capture probe and the enzyme from the array; contacting an analyte in the biological sample with a capture domain of a capture probe in an area of the array covered by the biological sample; and determining (i) the sequence of the spatial barcode of the capture probe or a complement thereof in the area of the array covered by the biological sample, and (ii) all or a portion of the sequence of the analyte bound to the capture domain of the capture probe or a complement thereof in the area of the array covered by the biological sample, and using the sequences of (i) and (ii) to determine the location of the analyte in the biological sample.

In some embodiments, the biological sample is a tissue sample. In some embodiments, the tissue sample is a tissue section. In some embodiments, the tissue section is a fresh, frozen tissue section. In some embodiments, the biological sample is a clinical sample. For example, the clinical sample can be selected from the group consisting of whole blood, blood-derived products, blood cells, and combinations thereof. In some embodiments, the clinical sample is a cultured tissue. In some embodiments, the clinical sample is cultured cells. In some embodiments, the clinical sample is a cell suspension. In some embodiments, the biological sample is an organoid, embryonic stem cells, pluripotent stem cells, and combinations thereof. In some embodiments, the organoid is selected from the group consisting of a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, a retinal organoid, and combinations thereof. In some embodiments, the biological sample includes diseased cells, fetal cells, immune cells, cellular macromolecules, organelles, extracellular polynucleotides, and combinations thereof.

In some embodiments, the biological sample on the array is imaged prior to cleaving the capture probe. Imaging the biological sample can indicate where the boundaries of the biological sample are and where the area not covered by the biological sample begins. In some embodiments, the capture probes on the array can be selectively cleaved such that only the capture probes in area(a) not covered by the biological sample are separated from the array. In some embodiments, an area of the array not covered by the biological sample is exposed to an enzyme that cleaves the cleavage sequence hybridized to the cleavage probe, thereby releasing capture probe(s) in an area of the array not covered by the biological sample.

In some embodiments, prior to cleaving the photocleavable linker, the biological sample on the array is imaged. In some embodiments, the capture probes on the array can be selectively cleaved (e.g., selectively photocleaved) such that only the capture probes in areas not covered by the biological sample are separated from the array. In some embodiments, a photocleavable linker can be selectively cleaved by protecting the biological sample from light and applying light to the array. In some embodiments, the area of the array not covered by the biological sample is exposed to a wavelength of light. In this way, the biological sample and the capture probes covered by the biological sample are protected, and the capture probes not covered by the biological sample (e.g., the area of the array outside of the biological sample) are photocleaved from the substrate.

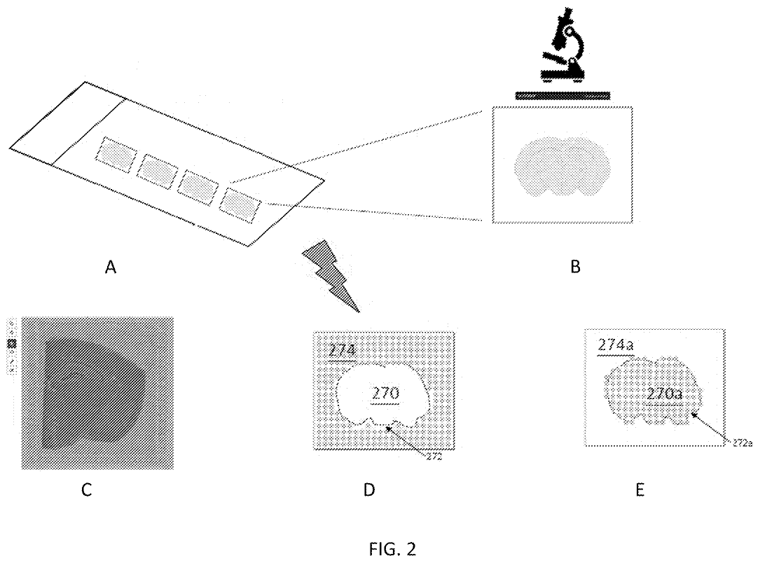

FIG. 2A-E illustrates an example workflow for cleaving capture probes not covered by a biological sample in accordance with the present disclosure. FIG. 2A shows four biological samples (e.g., tissue sections), respectively positioned on individual arrays including capture probes that include a photocleavable linker. While four biological samples and four arrays are shown in FIG. 2A, it is understood that there can be more or less than four biological samples and/or four arrays.

FIG. 2B shows an expanded view of a biological sample positioned on an array.

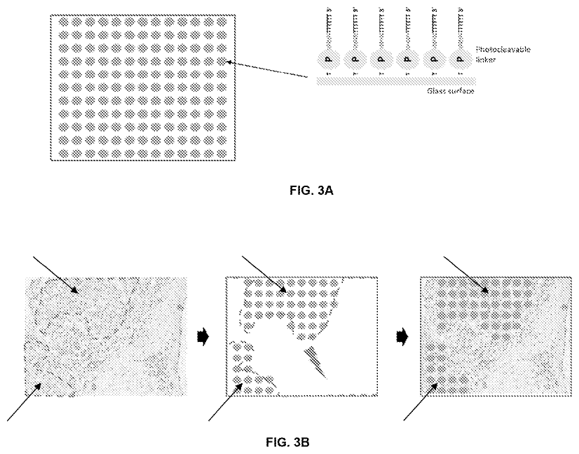

FIG. 3A is a schematic diagram showing an example of capture probes comprising photocleavable groups on the surface of an array, for example one of the four sections shown in FIG. 2A. FIG. 3B shows an exemplary workflow for cleaving capture probes from an area not covered by a biological sample. The left panel of FIG. 3B shows a biological sample (e.g., a tissue section) that is disposed on an array (e.g., an array that includes a plurality of capture probes coupled to a substrate via a photocleavable linker). While one biological sample and two regions of interest (indicated by arrows) are shown in the left panel of FIG. 3A, it is understood that there can be more or less than two regions of interest in a biological sample on a single array. The middle panel of FIG. 3B shows photocleavage of capture probes in areas of the array not covered by the biological sample. The right panel of FIG. 3B shows transfer of mRNA from the regions of interest (marked with arrows) in the biological sample to the areas of the array that maintained their capture probes after photocleavage; the regions of interest that were previously defined in the biological sample.

In some embodiments, the biological sample is imaged and/or stained. In some embodiments, imaging is performed using an imaging technique selected from the group comprising: expansion microscopy, bright field microscopy, dark field microscopy, phase contrast microscopy, electron microscopy, fluorescence microscopy, reflection microscopy, interference microscopy, confocal microscopy, and visual identification, and a combination thereof.

In some embodiments, an image of the boundaries of the array on which is placed the biological sample includes fiducials which are included on the substrate to allow for proper orientation, detection, and/or rotation of the biological sample on the substrate. In some embodiments, the imaging technique can include applying a phase contrast to the biological sample.

In some embodiments, imaging can include staining the biological sample. In some embodiments, the staining comprises a biological stain selected from the group consisting of: acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, and combinations thereof. In some embodiments, staining the biological sample comprises eosin and hematoxylin. In some embodiments, staining the biological sample comprises staining with a detectible label selected from the group consisting of: radioisotopes, fluorophores, chemiluminescent compounds, bioluminescent compounds, dyes, and combinations thereof.

In some embodiments, the biological sample on the array is analyzed after the biological sample has been stained and imaged. In some embodiments, the analysis determines which capture probes are present in an area covered by the biological sample and which capture probes are in an area of the array that is not covered by the biological sample. FIG. 2C shows an example of a stained and imaged biological sample positioned on an array comprising a plurality of capture probes.

In some embodiments, the capture probes on an area of the array outside of the biological sample (e.g., not covered by the biological sample) are the capture probes that are photocleaved and removed from the array. In some embodiments, prior to exposing the area of the array not covered by the biological sample to a wavelength of light, the biological sample (e.g., tissue section) can be analyzed to determine which capture probes are in an area of the array that is covered by the biological sample and which capture probes are in an area of the array that is not covered by the biological sample, as illustrated in FIG. 2D. As described herein, in some embodiments, the analysis of the biological sample can include analysis by a hardware and/or a software program to direct light to an area of the array not covered by the biological sample.

FIG. 2D shows a schematic of the biological sample 270 obscuring the capture probes on the area of the array underneath the biological sample 270 and a broken line illustrating a perimeter 272 of the biological sample. In this illustration, the capture probes on the array outside of the perimeter 272 of the biological sample 270 are represented as dots. The area of the array outside of the perimeter of the biological sample is labeled 274. In some embodiments, a light can be applied to the capture probes that are outside of the perimeter 272 of the biological sample 270 in area 274. For instance, a light can be selectively applied to the capture probes outside of the perimeter 272 of the biological sample 270 to photocleave only the probes that are in area 274. In this instance, the capture probes in area 274 can be removed as described herein.

FIG. 2E shows a schematic of the capture probes under the biological sample 270a and a broken line indicating the perimeter 272a of the biological sample 270a. In FIG. 2E the blank area 274a is the area outside the perimeter 272a of the biological sample 270a. FIG. 2E shows the area 274a of the array outside the perimeter 272a of the biological sample 270a with photocleaved capture probes cleaved off the array and removed (e.g., washed away). In this instance, the location of an analyte in the biological sample 270a includes a determination of the capture probes under the biological sample 270a. Upon removal of the capture probes in area 274a analyte capture in area 274a is greatly decreased or obliterated, thereby decreasing background and off target binding of analytes.

In some embodiments, a digital mirror device or DMD can be operated with a microscope (e.g., ANDOR MOSAIC 3®) to photocleave the capture probes in an area surrounding the biological sample. In some embodiments, the DMD and/or microscope can be operated with a computing device comprising a computer readable medium (e.g., hardware and/or software) storing instructions, that when executed by the computing device (e.g., one or more processing resources), cause it to perform (e.g., execute) the instruction for illumination of an area of an array. For example, a DMD and/or microscope can be communicatively coupled to a computing device including computer readable medium (e.g., a memory device, DRAM, SRAM, etc.). The memory device can be programmed with instructions (e.g., software) and/or hardware logic (e.g., FPGA, ASIC, etc.) to illuminate one or more areas on an array for a period of time at a defined wavelength. The memory device can store computer readable instructions that when executed by a computing device (e.g., a processing resource included in the computing device), the microscope and/or DMD will perform the instructions. In some embodiments, the instructions can include determining (e.g., detecting) a location of a biological sample on an array. In some embodiments, the instructions can include orientating the position of the array based on the determined (e.g., detected) location of the biological sample on the array. For example, the computing device (e.g., a processing resource on a computing device) can execute instructions to alter the position of a stage of the microscope based on the position of the biological sample positioned on the array on the stage.

In some embodiments, the instructions can include determining (e.g., detecting) the biological sample on the array based (at least in part) on an image of the biological sample on the array. For example, as described above, the biological sample can be imaged (e.g., stained). In some embodiments, the instructions can include analyzing the biological sample on the array after the biological sample has been stained and/or imaged. In some embodiments, analyzing can include instructions that when executed by a processing resource include determining the capture probes on the array that are under the biological sample and/or determining capture probes on the array that are outside of the area of the biological sample (e.g., not covered by the biological sample). In some embodiments, analyzing can include instructions that when executed by a processing resource include determining the capture probes on the array that are present in a region of interest in the biological sample and/or determining capture probes on the array that are outside the region of interest in the biological sample (e.g., not covered by the biological sample).

In some embodiments, the computing device can include instructions that when executed by a processing resource include determining an area of the array outside of the biological sample. In some embodiments, the computing device can include instructions that when executed by a processing resource include determining the capture probes on the array that are outside of the biological sample. In some embodiments, the computing device can include instructions that when executed by a processing resource include applying a light to the area outside of the biological sample or an area outside of the region of interest in the biological sample. For example, the computing device can execute instructions that cause the DMD (e.g., ANDOR MOSAIC 3®) to direct a light source to capture probes on the array that are outside of the biological sample or outside a region of interest in the biological sample. The light source can be precisely applied to capture probes that are outside of the biological sample or outside a region of interest in the biological sample. In this way, the biological sample or a region of interest in the biological sample is not exposed to the light source and the capture probes under the biological sample or under the region of interest in the biological sample are not exposed to the light source. In some embodiments, a mask can be applied to the biological sample prior to illumination in order to protect the biological sample from photodamage.

In some embodiments, the cleaved (e.g., photocleaved, enzymatically cleaved, etc.) capture probes in an area of the array not covered by the biological sample or not covered by a region of interest in the biological sample are removed by washing (e.g., using a wash buffer). In some embodiments, the wash buffer can be added to contact the area of the array not covered by the biological sample or the area of the array not covered by the region of interest in the biological sample, and the wash buffer (including the cleaved capture probes from the area of the array not covered by the biological sample) are removed by pipetting, wicking, or other methods known in the art. In some embodiments, a combination of removing methods can be used. Such removing (e.g., washing) methods can also be used for removing unhybridized cleavage probes. In some embodiments, washing and removing steps can be repeated (e.g., at least 2 times, 3 times, 4 times, or greater). In some embodiments, a drying step can be performed after washing (e.g., air dry).

In some embodiments, the wash buffer is added automatically (e.g., by a robot) or manually (e.g., by pipetting). In some embodiments, the wash buffer is added vertically above the area of the array that is outside of the biological sample (e.g., not covered by the biological sample) or outside of the region of interest in the biological sample (e.g., not covered by the region of interest in the biological sample). In some embodiments, the wash buffer is added dropwise by a pipette. In some embodiments, the wash buffer is added to contact all or a portion of the area of the array outside of the biological sample (e.g., not covered by the biological sample) or all or a portion of the area of the array outside of the region of interest in the biological sample (e.g., not covered by the region of interest in the biological sample). In some embodiments, the wash buffer is added to all or a portion of a surface of the biological sample that is contacting the array.

In some embodiments, the wash buffer is 1×TE buffer, 1×TAE buffer, 1×TBE buffer, or PBS. In some embodiments, the wash buffer contains a buffer (e.g., Tris, MOPS, HEPES, MES, or any other buffer known in the art), chelating agents (e.g., ethylenediaminetetraacetic acid (EDTA)) and/or metal ions (e.g., Mg²⁺). In some embodiments, the wash buffer can have a pH that is about 5.0, about 5.5, about 6.0, about 6.5, about 7.0, about 7.5, about 8.0, about 8.5, about 9.0, about 9.5, or about 10.0, or about 5.0 to 5.5, about 5.5 to 6.0, about 6.0 to 6.5, about 6.5 to 7.0, about 7.0 to 7.5, about 7.5 to 8.0, about 8.0 to 8.5, about 8.5 to 9.0, about 9.0 to 9.5, or about 9.5 to 10.0.

In some embodiments, the area of the array outside of the biological sample (e.g., not covered by the biological sample) or the area of the array outside of the region of interest in the biological sample is contacted by the wash buffer for about 5 seconds to about 1 hour, about 5 seconds to about 50 minutes, about 5 seconds to about 40 minutes, about 5 seconds to about 30 minutes, about 5 seconds to about 20 minutes, about 5 seconds to about 10 minutes, about 5 seconds to about 5 minutes, about 5 seconds to about 1 minute, about 5 seconds to about 30 seconds, about 5 seconds to about 10 seconds, about 10 seconds to about 1 hour, about 10 seconds to about 50 minutes, about 10 seconds to about 40 minutes, about 10 seconds to about 30 minutes, about 10 seconds to about 20 minutes, about 10 seconds to about 10 minutes, about 10 seconds to about 5 minutes, about 10 seconds to about 1 minute, about 10 seconds to about 30 seconds, about 30 seconds to about 1 hour, about 30 seconds to about 50 minutes, about 30 seconds to about 40 minutes, about 30 seconds to about 30 minutes, about 30 seconds to about 20 minutes, about 30 seconds to about 10 minutes, about 30 seconds to about 5 minutes, about 30 seconds to about 1 minute, about 1 minute to about 1 hour, about 1 minute to about 50 minutes, about 1 minute to about 40 minutes, about 1 minute to about 30 minutes, about 1 minute to about 20 minutes, about 1 minute to about 10 minutes, about 1 minute to about 5 minutes, about 5 minutes to about 1 hour, about 5 minutes to about 50 minutes, about 5 minutes to about 40 minutes, about 5 minutes to about 30 minutes, about 5 minutes to about 20 minutes, about 5 minutes to about 10 minutes, about 10 minutes to about 1 hour, about 10 minutes to about 50 minutes, about 10 minutes to about 40 minutes, about 10 minutes to about 30 minutes, about 10 minutes to about 20 minutes, about 20 minutes to about 1 hour, about 20 minutes to about 50 minutes, about 20 minutes to about 40 minutes, about 20 minutes to about 30 minutes, about 30 minutes to about 1 hour, about 30 minutes to about 50 minutes, about 30 minutes to about 40 minutes, about 40 minutes to about 1 hour, about 40 minutes to about 50 minutes, or about 50 minutes to about 1 hour at a temperature of about 4° C. to about 35° C., about 4° C. to about 30° C., about 4° C. to about 25° C., about 4° C. to about 20° C., about 4° C. to about 15° C., about 4° C. to about 10° C., about 10° C. to about 35° C. to about 10° C. to about 30° C., about 10° C. to about 25° C., about 10° C. to about 20° C., about 10° C. to about 15° C., about 15° C. to about 35° C., about 15° C. to about 30° C., about 15° C. to about 25° C., about 15° C. to about 20° C., about 20° C. to about 35° C., about 20° C. to about 30° C., about 20° to about 25° C., about 25° C. to about 35° C., about 25° C. to about 30° C., or about 30° C. to about 35° C.

In some embodiments, the area of the array that is outside of the biological sample (e.g., not covered by the biological sample) or the area of the array that is outside of the region of interest in the biological sample (e.g., not covered by the region of interest in the biological sample) can be contacted by the wash buffer for at least about 1 second, at least about 5 seconds, at least about 10 seconds, at least about 15 seconds, at least about 20 seconds, at least about 30 seconds, at least about 45 seconds, at least about 1 minutes, at least about 5 minutes, at least about 10 minutes, at least about 15 minutes, at least about 20 minutes, at least about 25 minutes, at least about 30 minutes, at least about 40 minutes, at least about 50 minutes, at least about 1 hour, at a temperature of about 4° C., about 10° C., about 20° C., about 25° C., about 30° C., about 32° C., about 34° C., about 35° C., about 36° C., about 37° C., about 38° C., about 39° C., about 40° C., about 45° C. or about 50° C.

Provided herein is a method for determining a location of an analyte in a biological sample, the method comprising (a) contacting a biological sample with an array comprising a plurality of capture probes comprising: (i) a photocleavable linker, (ii) a spatial barcode, and (iii) a capture domain, where the capture domain binds to the analyte, and (iii), (b) exposing the area of the array not covered by the biological sample with a wavelength of light that cleaves the photocleavable linker of the capture probes, thereby releasing the capture probes from the area of the array not covered by the biological sample, (c) removing the cleaved capture probes, (d) contacting an analyte in the biological sample with a capture domain of a capture probe covered by the biological sample; and (e) determining (i) the sequence of the spatial barcode of the capture probe or a complement thereof in an area of the array covered by the biological sample, and (ii) all or a portion of the sequence of the analyte bound to the capture domain of the capture probe or a complement thereof in the area of the array covered by the biological sample, and using the sequences of (i) and (ii) to determine the location of the analyte in the biological sample.

Also provided herein is a method of decreasing hybridization of analytes in a biological sample to capture probes in an area of an array that is not covered by the biological sample, where the method comprises: (a) contacting the biological sample with the array, wherein the array comprises a plurality of capture probes comprising (i) a photocleavable linker; (ii) a spatial barcode, and (iii) a capture domain; and (b) exposing the area of the array not covered by the biological sample with a wavelength of light that cleaves the photocleavable linker of the capture probes, thereby releasing the capture probes from the area of the array not covered by the biological sample, thereby decreasing hybridization of analytes in the biological sample to capture probes in the area of the array that is not covered by the biological sample.

Alternatively, the location of an analyte in a biological sample can be determined by: (a) contacting a biological sample with an array comprising a plurality of capture probes, wherein each capture probe of the plurality of capture probes comprises (i) a capture domain, (ii) a cleavage sequence, and (iii) a spatial barcode; (b) contacting the array with a plurality of cleavage probes, wherein a cleavage probe of the plurality of cleavage probes hybridizes to a cleavage sequence in a capture probe in an area of the array not covered by the biological sample; (c) contacting the area of the array not covered by the biological sample with an enzyme that cleaves the cleavage sequence/cleavage probe hybrid, thereby releasing the capture probe from the area of the array not covered by the biological sample; (d) removing the cleaved capture probes and the enzyme from the array; (e) contacting an analyte in the biological sample with a capture domain of a capture probe in an area of the array covered by the biological sample; and (f) determining (i) the sequence of the spatial barcode of the capture probe in the area of the array covered by the biological sample, or a complement thereof, and (ii) all or a portion of the sequence of the analyte bound to the capture domain of the capture probe in the area of the array covered by the biological sample, or a complement thereof, and using the sequences of (i) and (ii) to determine the location of the analyte in the biological sample. In some embodiments, the method can include permeabilizing the biological sample (e.g., using any of the methods of permeabilization described herein).

Also provided herein is a method of decreasing hybridization of analytes in a biological sample to capture probes in an area of an array that is not covered by the biological sample, where the method comprises: (a) contacting the biological sample with the array, wherein the array comprises a plurality of capture probes, wherein each capture probe of the plurality of capture probes comprises (i) a capture domain, (ii) a cleavage sequence, and (iii) a spatial barcode, wherein the cleavage sequence is positioned 5′ relative to the capture domain and the 5′ end of the capture probe is attached to a substrate; (b) contacting the array with a plurality of cleavage probes, wherein a cleavage probe of the plurality of cleavage probes hybridizes to a cleavage sequence in a capture probe in an area of the array not covered by the biological sample; and (c) contacting the area of the array not covered by the biological sample with an enzyme that cleaves the cleavage sequence hybridized to the cleavage probe, thereby releasing the capture probe from the area of the array not covered by the biological sample, thereby decreasing hybridization of analytes in the biological sample to capture probes in the area of the array that is not covered by the biological sample.

In some embodiments, permeabilizing the biological sample is performed before contacting the analyte with the capture domain of the capture probes. In some embodiments, the capture probes of the plurality of capture probes under the biological sample on the array can be extended. For instance, in some embodiments, the method comprises extending a 3′ end of the capture probe using the analyte, a ligation production, or an analyte capture agent as an extension template.

In some embodiments, the analyte(s) is/are DNA. In some embodiments, the analyte(s) is/are genomic DNA. In some embodiments, the analyte(s) is/are RNA. In some embodiments, the RNA is mRNA. In some embodiments, the analyte(s) in the biological sample is/are a non-polyadenylated nucleic acid.

In some embodiments, when an analyte in the biological sample is a non-polyadenylated nucleic acid, nucleotide templated ligation can detect a non-polyadenylated analyte in the biological sample. Nucleotide templated ligation is a process that includes multiple probes (e.g., oligonucleotides or oligonucleotide probes) that hybridize to adjacent complementary analyte sequences. Upon hybridization, the two probes are ligated to one another, creating a ligation product only in the event that both probes hybridize to their respective complementary sequences. In some instances, at least one of the probes includes a sequence (e.g., a poly-adenylated sequence) that can bind (e.g., hybridize) a capture domain of a capture probe on an array described herein (e.g., the capture domain comprises a poly(T) sequence in some instances). In some instances, prior to hybridization of the poly(T) to the poly(A) sequence, a nuclease (e.g., an endonuclease) digests the analyte (e.g., nucleic acid) hybridized to the ligation product. The digestion step releases the ligation product to hybridize to the capture domain of the capture probe. In this way, nucleotide templated ligation provides a method to perform targeted capture of non-polyadenylated nucleic acids.

In some embodiments, after removing the one or more photocleaved or cleaved capture probes from the area of the array not covered by the biological sample as described herein, the method includes hybridizing a probe set to the analyte (e.g., a non-polyadenylated nucleic acid).

In some embodiments, the analyte is a non-polyadenylated nucleic acid in the biological sample. In some embodiments, methods here further comprise contacting the analyte with a first probe and a second probe, where the first probe comprises a sequence that hybridizes to a first sequence in a nucleic acid in the biological sample, and the second probe comprises (i) a sequence that hybridizes to a second sequence in the nucleic acid in the biological sample, and (ii) a sequence that binds the capture domain of the capture probe in the area of the array covered by the biological sample, and ligating the first probe and the second probe to generate the ligation product, where the ligation product binds the capture domain of the capture probe in the area of the array covered by the biological sample.

In some embodiments, the determining step comprises determining the sequence of (i) the spatial barcode of the capture probe in the area of the array covered by the biological sample, or a complement thereof, and (ii) all or a portion of the analyte bound to the capture domain of the capture probe in the area of the array covered by the biological sample, or a complement thereof. In some embodiments, the sequencing is high-throughput sequencing.

Also provided herein is a method for determining a location of an analyte in a biological sample, the method comprising, (a) contacting a biological sample with an array comprising a plurality of capture probes (i) a photocleavable linker; (ii) a spatial barcode, and (iii) a capture domain, (b) contacting a plurality of analyte capture agents with the biological sample comprising (i) an analyte capture sequence that hybridizes to the capture domains of the plurality of capture probes, (ii) an analyte binding moiety barcode, and (iii) an analyte binding moiety that binds the analyte, (c) exposing the area of the array not covered by the biological sample with a wavelength of light that cleaves the photocleavable linker of the capture probes, thereby releasing the capture probes from the area of the array not covered by the biological sample, (d) removing the cleaved capture probes, (e) contacting an analyte capture agent with a capture domain of a capture probe in an area of the array covered by the biological sample, and (f) determining (i) the sequence of the spatial barcode of the capture probe in the area of the array covered by the biological sample, or a complement thereof, and (ii) all or a portion of the sequence of the analyte binding moiety barcode, or a complement thereof, and using the sequences of (i) and (ii) to identify the location of the analyte in the biological sample.

Also provided herein is a method of decreasing hybridization of analyte capture agents to capture probes in an area of an array that is not covered by the biological sample, wherein the method comprises: (a) contacting the biological sample with the array, wherein the array comprises a plurality of capture probes comprising (i) a photocleavable linker; (ii) a spatial barcode, and (iii) a capture domain; (b) contacting the analyte capture agents with the biological sample, wherein the analyte capture agents comprise (i) an analyte capture sequence that hybridizes to the capture domains of the plurality of capture probes, (ii) an analyte binding moiety barcode, and (iii) an analyte binding moiety that binds to an analyte; and (c) exposing the area of the array that is not covered by the biological sample with a wavelength of light that cleaves the photocleavable linker of the capture probes, thereby releasing the capture probes from the area of the array not covered by the biological sample, thereby decreasing hybridization of analyte capture agents to capture probes in the area of the array that is not covered by the biological sample.

Alternatively, location of an analyte in a biological sample can be determined by: (a) contacting a biological sample with an array comprising a plurality of capture probes, wherein each capture probe of the plurality of capture probes comprises (i) a capture domain, (ii) a cleavage sequence, and (iii) a spatial barcode; (b) contacting a plurality of analyte capture agents with the biological sample, wherein each analyte capture agent of the plurality of analyte capture agents comprises (i) an analyte capture sequence that hybridizes to the capture domain of the plurality of capture probes, (ii) an analyte binding moiety barcode, and (iii) an analyte binding moiety that binds the analyte; (c) contacting the array with a plurality of cleavage probes, wherein a cleavage probe of the plurality of cleavage probes hybridizes to a cleavage sequence in a capture probe in an area of the array not covered by the biological sample; (d) exposing the area of the array not covered by the biological sample with an enzyme that cleaves the cleavage sequence/cleavage probe hybrid, thereby releasing the capture probe from the area of the array not covered by the biological sample; (e) removing the cleaved capture probe and the enzyme from the array; (f) contacting an analyte capture agent with a capture domain of a capture probe in an area of the array covered by the biological sample; and (g) determining (i) the sequence of the spatial barcode of the capture probes in the area of the array covered by the biological sample, or a complement thereof, and (ii) all or a portion of the sequence of the analyte binding moiety barcode, or a complement thereof, and using the sequences of (i) and (ii) to determine the location of the analyte in the biological sample.

Also provided herein is a method of decreasing hybridization of analyte capture agents to capture probes in an area of an array that is not covered by the biological sample, wherein the method comprises: (a) contacting the biological sample with the array, wherein the array comprises a plurality of capture probes, wherein each capture probe of the plurality of capture probes comprises (i) a capture domain, (ii) a cleavage sequence, and (iii) a spatial barcode, wherein the cleavage sequence is positioned 5′ relative to the capture domain and the 5′ end of the capture probe is attached to a substrate; (b) contacting a plurality of analyte capture agents with the biological sample, wherein each analyte capture agent of the plurality of analyte capture agents comprises (i) an analyte capture sequence that hybridizes to the capture domains of the plurality of capture probes, (ii) an analyte binding moiety barcode, and (iii) an analyte binding moiety that binds the analyte; (c) contacting the array with a plurality of cleavage probes, wherein a cleavage probe of the plurality of cleavage probes hybridizes to a cleavage sequence in a capture probe in an area of the array not covered by the biological sample; and (d) contacting the area of the array not covered by the biological sample with an enzyme that cleaves the cleavage sequence hybridized to the cleavage probe, thereby releasing the capture probe from the area of the array not covered by the biological sample, thereby decreasing hybridization of analyte capture agents to capture probes in the area of the array that is not covered by the biological sample. In some embodiments, the method can further comprise permeabilizing the biological sample (e.g., using any of the methods of permeabilization described herein). In some embodiments, permeabilizing the biological sample is performed before contacting the analyte capture agent with the biological sample.

In some embodiments, the determining step comprising sequencing (i) the sequence of the spatial barcode, or a complement thereof, of the capture probe in the area of the array covered by the biological sample, and (ii) all or a portion of the sequence corresponding to the analyte binding moiety barcode, or a complement thereof, and using the sequences of (i) and (ii) to determine the location of the analyte in the biological sample. In some instances, the sequencing is high throughput sequencing.

In some embodiments, the method further comprises extending a 3′ end of the capture probe of the plurality of capture probes under the biological sample using the analyte capture agent (e.g., the analyte binding moiety barcode of the analyte capture agent) as an extension template.

In some embodiments, the analyte(s) is/are a protein. In some embodiments, the protein(s) is/are intracellular protein(s). In some embodiments, the protein(s) is/are extracellular protein(s). In some embodiments, the analyte binding moiety is an antibody or an antigen-binding moiety thereof

In some embodiments, contacting the biological sample with an array comprising a plurality of capture probes, and contacting the plurality of analyte capture agents with the biological sample are performed at the same time.

In some embodiments, contacting the biological sample with an array comprising a plurality of capture probes is performed before contacting the plurality of analyte capture agents with the biological sample.

In some embodiments, contacting a plurality of analyte capture agents with the biological sample is performed before contacting the biological sample with an array comprising the plurality of capture probes.

In some embodiments of any of the methods described herein, the array comprising a plurality of capture probes, where a capture probe of the plurality of capture probes comprises: (i) a photocleavable linker, (ii) a spatial barcode, (iii) a capture domain that binds an analyte capture sequence, and one or more features. For example, the capture probes are coupled to a feature via a photocleavable linker. In some instances, the one or more features comprises a bead.

In some embodiments, of any of the methods described herein, an array can be exposed to a wavelength of light to cleave photocleavable capture probes while the biological sample is protected from the light. In some embodiments, the biological sample can be protected by a photomask. A photomask can be positioned on the array such that only specific regions of the array are exposed to light.

In some embodiments of any of the methods described herein, light can be applied to the capture probes in an area of the array that is not covered by the biological sample or an area of the array that is not covered by a region of interest in the biological sample. In some embodiments, a light can be directed to the area of the array that is not covered by the biological sample or the area of the array that is not covered by the region of interest in the biological sample by a mirror, a mirror array, a lens, or a moving stage. In some embodiments, a scanning laser contacts the area of the array that is not covered by the biological sample or the area of the array that is not covered by the region of interest in the biological sample thereby cleaving the photocleavable linker of the one or more capture probes on the area of the array that is not covered by the biological sample or the area of the array that is not covered by the region of interest in the biological sample. In some embodiments, the cleaving the photocleavable linker comprises heat. In some embodiments, cleaving the photocleavable linker comprises UV light.

### Exemplary Systems

The methods described above for analyzing biological samples can be implemented using a variety of hardware components. In this section, examples of such components are described. However, it should be understood that in general the various steps and techniques discussed herein can be performed using a variety of different devices and system components, not all of which are expressly set forth.

The systems described in this section include components that are specifically adapted for use in the methods described herein. In particular, the systems include light sources that can be directed to different portions of an array to cleave photocleavable linkages to release capture probes from the array so they do not interact with analytes from a biological sample, such as capture probes that are positioned outside a boundary region on the array that is defined by the edges of a sample that contacts the array. Light sources that can selectively direct light (e.g., UV light) to cleave probe linkages include, but are not limited to, directed light sources (e.g., digital mirror devices) as described above. MEMS-based digital mirror devices, for example, can be used for this purpose.

Other suitable light sources that can be used in the systems described below include light sources for which the spatial distribution of light emitted by the light source is modifiable. Suitable components for modifying the spatial distribution of light include, but are not limited to, photomasks, apertures, and other static mask components, and a wide variety of dynamic light modulators such as spatial light modulators. Examples of spatial light modulators are described for example in U.S. Pat. Nos. 6,348,990, 6,157,432, and 7,561,336, the entire contents of each of which are incorporated herein by reference.

The methods described herein can also include cleavage of linkages in analyte capture probes using cleavage probes and enzymes. In such methods, a cleavage probe is delivered to the array and hybridizes to a cleavage sequence in the analyte capture probe. An enzyme is then delivered to the array and cleaves the analyte capture probe at the location of the cleavage sequence/cleavage probe complex. The systems described herein include reservoirs for storing reagents such as cleavage probes and enzymes, and fluid handling components (e.g., channels, pumps, valves, and other components for regulating fluid flow) for delivering cleavage probes and enzymes to array locations to cleave analyte capture probes. As will be discussed in greater detail below, the components can be configured to selectively deliver such reagents to locations on the array that are outside the boundary region on the array that is defined by the edges of a sample that contacts the array, so that only analyte capture probes that are outside the boundary are cleaved.

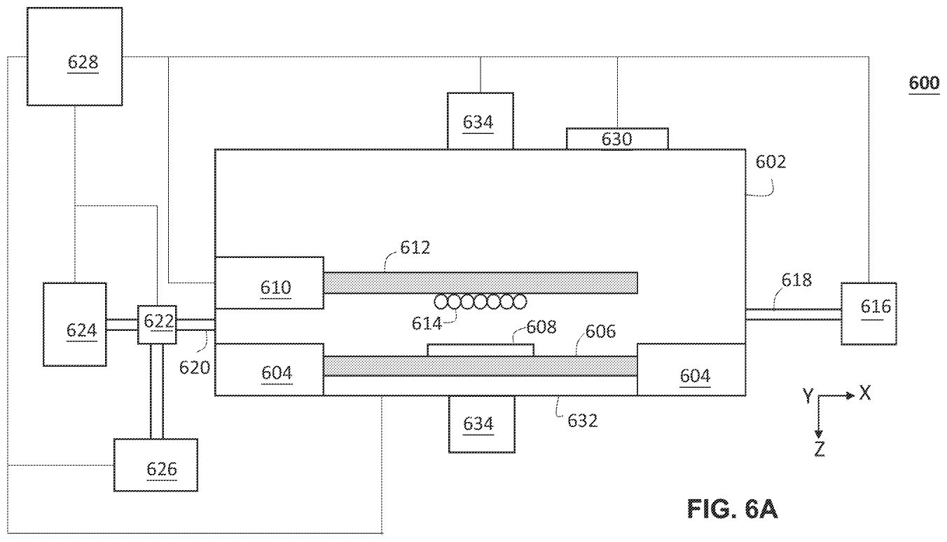

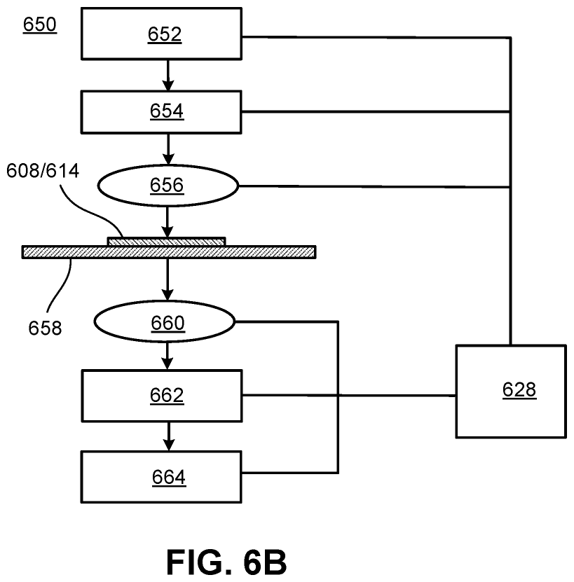

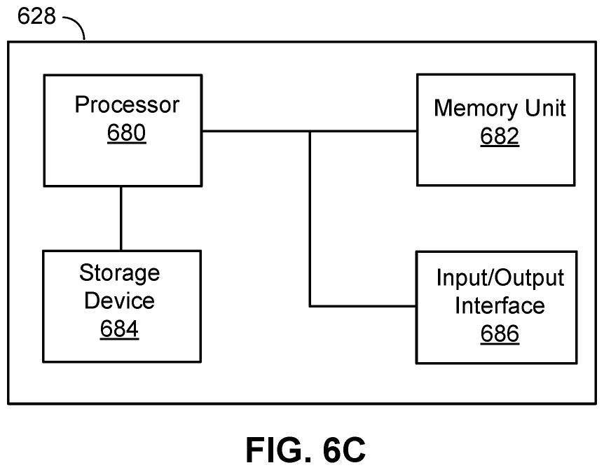

In general, a wide variety of different systems and components can be used to perform the methods and steps described herein. FIG. 6A is a schematic diagram showing an example sample handling apparatus 600. Sample handling apparatus 600 includes a sample chamber 602 that, when closed or sealed, is fluid-tight. Within chamber 602, a first holder 604 holds a first substrate 606 on which a biological sample 608 is positioned. Sample chamber 602 also includes a second holder 610 that holds a second substrate 612 with an array of features 614, as described above.

A fluid reservoir 616 is connected to the interior volume of sample chamber 602 via a fluid inlet 618. Fluid outlet 620 is also connected to the interior volume of sample chamber 602, and to valve 622. In turn, valve 622 is connected to waste reservoir 624 and, optionally, to analysis apparatus 626. A control unit 628 is electrically connected to second holder 610, to valve 622, to waste reservoir 624, and to fluid reservoir 616.

During operation of apparatus 600, any of the reagents, solutions, and other biochemical components described above can be delivered into sample chamber 602 from fluid reservoir 616 via fluid inlet 618. Control unit 628, connected to fluid reservoir 616, can control the delivery of reagents, solutions, and components, and adjust the volumes and flow rates according to programmed analytical protocols for various sample types and analysis procedures. In some embodiments, fluid reservoir 616 includes a pump, which can be controlled by control unit 628, to facilitate delivery of substances into sample chamber 602.

In certain embodiments, fluid reservoir 616 includes a plurality of chambers, each of which is connected to fluid inlet 618 via a manifold (not shown). Control unit 628 can selectively deliver substances from any one or more of the multiple chambers into sample chamber 602 by adjusting the manifold to ensure that the selected chambers are fluidically connected to fluid inlet 618.

In general, control unit 628 can be configured to introduce substances from fluid reservoir 616 into sample chamber 602 before, after, or both before and after, sample 608 on first substrate 606 has interacted with the array of features 614 on first substrate 612. Many examples of such substances have been described previously. Examples of such substances include, but are not limited to, permeabilizing agents, buffers, fixatives, staining solutions, washing solutions, and solutions of various biological reagents (e.g., enzymes, peptides, oligonucleotides, primers).

To initiate interaction between sample 608 and feature array 614, the sample and array are brought into spatial proximity. To facilitate this step, second holder 610—under the control of control unit 628—can translate second substrate 612 in any of the x-, y-, and z-coordinate directions. In particular, control unit 628 can direct second holder 610 to translate second substrate 612 in the z-direction so that sample 608 contacts, or nearly contacts, feature array 614.

In some embodiments, apparatus 600 can optionally include an alignment sub-system 630, which can be electrically connected to control unit 628. Alignment sub-system 630 functions to ensure that sample 608 and feature array 614 are aligned in the x-y plane prior to translating second substrate 612 in the z-direction so that sample 608 contacts, or nearly contacts, feature array 614.

Alignment sub-system 630 can be implemented in a variety of ways. In some embodiments, for example, alignment sub-system 630 includes an imaging unit that obtains one or more images showing fiducial markings on first substrate 606 and/or second substrate 612. Control unit 628 analyzes the image(s) to determine appropriate translations of second substrate 612 in the x- and/or y-coordinate directions to ensure that biological sample 608 and feature array 614 are aligned prior to migration of analytes in the biological sample 608 to the feature array 614.

Control unit 628 can also direct the system to analyze the image(s) to identify one or more regions of interest (ROIs) within the biological sample. As discussed above, the outer edge of the biological sample defines a boundary on the array. Features located outside the boundary include analyte capture probes that can interact with analytes released from the sample, however such interactions are unwanted and lead to a decrease in assay resolution. The analyte capture probes can be released from the array (e.g., via cleavage of linkers or sequences included in the capture probes), thereby mitigating the capture of analytes to capture probes outside and around the tissue. Alternatively, once regions of interest are determined and spatially identified the information is used by control unit 628 to direct light and/or reagents to locations on the array that are outside the ROIs to release analyte capture probes from the array at those locations to mitigate binding of analytes to the capture probes not in the region on interest.

In certain embodiments, control unit 628 can optionally regulate the removal of substances from sample chamber 602. For example, control unit 628 can selectively adjust valve 622 so that substances introduced into sample chamber 602 from fluid reservoir 616 are directed into waste reservoir 624. In some embodiments, waste reservoir 624 can include a reduced-pressure source (not shown) electrically connected to control unit 628. Control unit 628 can adjust the fluid pressure in fluid outlet 620 to control the rate at which fluids are removed from sample chamber 602 into waste reservoir 624.

In some embodiments, amplification products or amplicons (e.g., a sequencing library) generated from feature array 614 can be selectively delivered to analysis apparatus 626 via suitable adjustment of valve 622 by control unit 628. As described above, in some embodiments, analysis apparatus 626 includes a reduced-pressure source (not shown) electrically connected to control unit 628, so that control unit 628 can adjust the rate at which analytes are delivered to analysis apparatus 626. As such, fluid outlet 620 effectively functions as an amplification product or amplicon (e.g., a sequencing library) collector, while analysis of the amplification products or amplicons (e.g., a sequencing library) is performed by analysis apparatus 626. It should be noted that not all of the workflows and methods described herein are implemented via analysis apparatus 626. For example, in some embodiments, analytes that are captured by feature array 614 remain bound to the array (i.e., are not cleaved from the array), and feature array 614 is directly analyzed to identify specifically-bound sample analytes.

In certain embodiments, reagents that include a cleavage probe and one or more enzymes are delivered to array 614 to cleave analyte capture ptobes from the array that are outside the one or more identified ROIs, which can correspond to the boundary/boundaries defined by the outer edges of the biological sample when the sample contacts the array or by one or more defined areas of a biological sample that are deemed to be of interest over other areas of a biological sample (ROIs). To deliver such reagents, the system can include an adjustable delivery mechanism (not shown in FIG. 6A) connected to fluid inlet 618 and coupled to control unit 628. Control unit 628 is configured to control the delivery mechanism to deliver reagents selectively to certain regions of array 614, i.e., the regions of array 614 that are outside the ROIs.

A wide variety of different adjustable delivery mechanisms can be used in analysis apparatus 626 to selectively deliver reagents to locations on array 614. Examples of such mechanisms include, but are not limited to, jet-based printing mechanisms as described in U.S. Pat. No. 10,724,078, mechanisms that use hollow-tip electrodes as described in U.S. Pat. No. 7,456,012, and mechanisms that use fluidic channels, e.g., in an array or network, as described in U.S. Patent Application Publication No. 2019/0099754. The entire contents of each of the foregoing references are incorporated by reference herein.

In addition to the components described above, apparatus 600 can optionally include other features as well. In some embodiments, for example, sample chamber 602 includes a heating sub-system 632 electrically connected to control unit 628. Control unit 628 can activate heating sub-system 632 to heat sample 608 and/or feature array 614, which can help to facilitate certain steps of the methods described herein.

In certain embodiments, sample chamber 602 includes an electrode 634 electrically connected to control unit 628. Control unit 628 can optionally activate electrode 634, thereby establishing an electric field between the first and second substrates. Such fields can be used, for example, to facilitate migration of analytes from sample 608 toward feature array 614.

In some of the methods described herein, one or more images of a sample and/or a feature array are acquired. As described above, in some embodiments, alignment mechanism 630 includes an imaging unit that is coupled to control unit 628 and used to obtain one or more images of sample 608 and/or array 614. The imaging unit of alignment mechanism 630 can generally include any of the components described below.

Alternatively, the systems described herein can include a separate imaging apparatus that is coupled to control unit 628 and used to obtain one or more images of the biological sample 608 and/or array 614. The imaging apparatus can generally be implemented in a variety of ways. FIG. 6B shows one example of an imaging apparatus 650. Imaging apparatus 650 includes a light source 652, light conditioning optics 654, light delivery optics 656, light collection optics 660, light adjusting optics 662, and a detection sub-system 664. Each of the foregoing components can optionally be connected to control unit 628, or alternatively, to another control unit. For purposes of explanation below, it will be assumed that control unit 628 is connected to the components of imaging apparatus 650.

During operation of imaging apparatus 650, light source 652 generates light. In general, the light generated by source 652 can include light in any one or more of the ultraviolet, visible, and/or infrared regions of the electromagnetic spectrum. A variety of different light source elements can be used to generate the light, including (but not limited to) light emitting diodes, laser diodes, laser sources, fluorescent sources, incandescent sources, and glow-discharge sources.

The light generated by light source 652 is received by light conditioning optics 654. In general, light conditioning optics 654 modify the light generated by light source 652 for specific imaging applications. For example, in some embodiments, light conditioning optics 654 modify the spectral properties of the light, e.g., by filtering out certain wavelengths of the light. For this purpose, light conditioning optics 654 can include a variety of spectral optical elements, such as optical filters, gratings, prisms, and chromatic beam splitters.

In certain embodiments, light conditioning optics 654 modify the spatial properties of the light generated by light source 652. For example, light conditioning optics 654 can include one or more optical elements or other components that can be adjusted (e.g., by control unit 628) to direct light generated by source 652 to a plurality of spatial locations. The plurality of spatial locations can include, for example, a plurality of locations on an array of features, a plurality of locations on a slide or other substrate supporting a sample, and where an array supports a sample, locations (i.e., features) on the array that are not covered by the biological sample or are outside of defined regions of interest on a biological sample.

Examples of components that can be used for this purpose include (but are not limited to) mirrors, masks, apertures, phase masks, apodizing elements, and diffusers. For example, in some embodiments, control unit 628 can adjust one or more mirrors of light conditioning optics 654 to direct light generated by source 652 to selected locations on an array of features. In certain embodiments, control unit 628 can adjust a position of one or more masks of light conditioning optics 654 to selectively direct light generated by source 652 to certain locations on an array of features.

After modification by light conditioning optics 654, the light is received by light delivery optics 656 and directed onto sample 608 or feature array 614, either of which is positioned on a mount 658. Light conditioning optics 654 generally function to collect and direct light onto the surface of the sample or array. A variety of different optical elements can be used for this purpose, and examples of such elements include, but are not limited to, lenses, mirrors, beam splitters, and various other elements having non-zero optical power.

Light emerging from sample 608 or feature array 614 is collected by light collection optics 660. In general, light collection optics 660 can include elements similar to any of those described above in connection with light delivery optics 656. The collected light can then optionally be modified by light adjusting optics 662, which can generally include any of the elements described above in connection with light conditioning optics 654.

The light is detected by detection sub-system 664. Generally, detection sub-system 664 functions to generate one or more images of sample 608 or feature array 614 by detecting light from the sample or feature array. A variety of different imaging elements can be used in detection sub-system 664, including CCD detectors and other image capture devices.

Each of the foregoing components can optionally be connected to control unit 628 as shown in FIG. 6B, so that control unit 628 can adjust various properties of the imaging apparatus. For example, control unit 628 can adjust the position of sample 608 or feature array 614 relative to the position of the incident light, and also with respect to the focal plane of the incident light (if the incident light is focused). Control unit 628 can also selectively filter both the incident light and the light emerging from the sample.

Imaging apparatus 650 can typically obtain images in a variety of different imaging modalities. In some embodiments, for example, the images are transmitted light images, as shown in FIG. 6B. In certain embodiments, apparatus 650 is configured to obtain reflection images. In some embodiments, apparatus 650 can be configured to obtain birefringence images, fluorescence images, phosphorescence images, multiphoton absorption images, and more generally, any known image type.

As described above, in some embodiments, one or more ROIs can be determined by control unit 628 based on one or more images of the biological sample 608 and/or array 614, i.e., when the biological sample contacts the array. The images can be acquired using imaging apparatus 650. To identify features on an array of features that are not covered by a tissue sample when a tissue sample is positioned over a subset of the array features, control unit 628 can direct detection subsystem 664 to obtain an image of the tissue section on the array, and control unit 628 can then identify features of the array that are not covered by the tissue section (e.g., by detecting differences transmitted light intensity between array features that are covered by the tissue section and those that are not covered).

In some embodiments of the methods described herein, photocleavable linkers of analyte capture probes are cleaved by light (e.g., ultraviolet light) generated by light source 652. When ROIs of array 614 are identified, analyte capture probes on the array that are outside the ROIs can be selectively illuminated and exposed to light to cleave photocleavable linkers in the capture probes, followed by removal of the cleaved and released the capture probes from array 614 prior to analysis.

With ROIs determined as described above, control unit 628 controls light source 652,. light conditioning optics 654, and/or light delivery optics 656 to selectively deliver light to illuminate regions of array 614 that are not within the ROIs. Light can be selectively delivered in a variety of ways. In some embodiments, one or more of light source 652, light conditioning optics 654, and light delivery optics 656 includes one or more masks, apertures, or other elements that selectively block certain portions of light generated by light source 652. The blocked portions of the light are typically the portions that would otherwise expose the ROIs on array 614.

In certain embodiments, one or more of light source 652, light conditioning optics 654, and light delivery optics 656 includes a configurable spatial light modulator coupled to control unit 628. Control unit 628 can adjust the spatial light modulator to control the spatial distribution of light that is incident on array 614, thereby selectively illuminating only regions of array 614 that are outside the ROIs to cleave analyte capture probes from the array. Suitable examples of spatial light modulators are provided above.

In some embodiments, light source 652, light conditioning optics 654, and light delivery optics 656 include optical elements that effectively function collectively as a directed light source such as a scanning light source. For example, light source 652 can include a laser source, and light conditioning optics 654 and/or light delivery optics 656 can include one or more mirrors or other reflective elements that can be adjusted by control unit 628 to selectively direct the light generated by light source 652 to different portions of array 614. Control unit 628 adjusts the mirrors and/or other reflective elements to direct the light to portions of array 614 that are outside the ROIs.

In certain embodiments, light source 652, light conditioning optics 654, and/or light delivery optics 656 include components that function as a digital mirror device (DMD), such as a MEMS-based DMD. Examples of suitable DMDs are described above,

In general, control unit 628 can perform any of the method steps described herein that do not expressly require user intervention by transmitting suitable control signals to the components of sample handling apparatus 600 and/or imaging apparatus 650. To perform such steps, control unit 628 generally includes software instructions that, when executed, cause control unit 628 to undertake specific steps. In some embodiments, control unit 628 includes an electronic processor and software instructions that are readable by the electronic processor, and cause the processor to carry out the steps described herein. In certain embodiments, control unit 628 includes one or more application-specific integrated circuits having circuit configurations that effectively function as software instructions.

Control unit 628 can be implemented in a variety of ways. FIG. 6C is a schematic diagram showing one example of control unit 628, including an electronic processor 680, a memory unit 682, a storage device 684, and an input/output interface 686. Processor 680 is capable of processing instructions stored in memory unit 682 or in storage device 684, and to display information on input/output interface 686.

Memory unit 682 stores information. In some embodiments, memory unit 682 is a computer-readable medium. Memory unit 682 can include volatile memory and/or non-volatile memory. Storage device 684 is capable of providing mass storage, and in some embodiments, is a computer-readable medium. In certain embodiments, storage device 684 may be a floppy disk device, a hard disk device, an optical disk device, a tape device, a solid state device, or another type of writeable medium.

The input/output interface 686 implements input/output operations. In some embodiments, the input/output interface 686 includes a keyboard and/or pointing device. In some embodiments, the input/output interface 686 includes a display unit for displaying graphical user interfaces and/or display information.

Instructions that are executed and cause control unit 628 to perform any of the steps or procedures described herein can be implemented in digital electronic circuitry, or in computer hardware, firmware, or in combinations of these. The instructions can be implemented in a computer program product tangibly embodied in an information carrier, e.g., in a machine-readable storage device, for execution by a programmable processor (e.g., processor 680). The computer program can be written in any form of programming language, including compiled or interpreted languages, and it can be deployed in any form, including as a stand-alone program or as a module, component, subroutine, or other unit suitable for use in a computing environment. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

Processor 680 can include any one or more of a variety of suitable processors. Suitable processors for the execution of a program of instructions include, by way of example, both general and special purpose microprocessors, and the sole processor or one of multiple processors of any kind of computer or computing device.

1-143. (canceled)

144. A method of decreasing hybridization of analytes from a biological sample to capture probes in an area of an array that is not covered by the biological sample, the method comprising:
(a) contacting the biological sample with the array, wherein the array comprises a plurality of capture probes, wherein a capture probe comprises: (i) a cleavage domain; (ii) a spatial barcode, and (iii) a capture domain; and
(b) cleaving the cleavage domain of the plurality of capture probes not covered by the biological sample, thereby releasing the plurality of capture probes from the area of the array not covered by the biological sample and decreasing hybridization of analytes from the biological sample in the area of the array not covered by the biological sample.

145. The method of claim 144, wherein the method further comprises imaging the biological sample on the array, wherein the imaging comprises one or more of expansion microscopy, bright field microscopy, dark field microscopy, phase contrast microscopy, electron microscopy, fluorescence microscopy, reflection microscopy, interference microscopy, confocal microscopy, and visual identification, or any combination thereof

146. The method of claim 144, further comprises staining the biological sample, wherein the staining comprises the use of acridine orange, Bismarck brown, carmine, coomassie blue, cresyl violet, DAPI, eosin, ethidium bromide, acid fuchsine, eosin and hematoxylin, Hoechst stains, iodine, methyl green, methylene blue, neutral red, Nile blue, Nile red, osmium tetroxide, propidium iodide, rhodamine, safranin, or any combination thereof or
the staining comprises the use of radioisotopes, fluorophores, chemiluminescent compounds, bioluminescent compounds, dyes, and combinations thereof.

147. The method of claim 144, wherein the cleavage domain is a photocleavable linker.

148. The method of claim 147, wherein releasing the plurality of capture probes comprises exposing the area of the array not covered by the biological sample with a wavelength of light from about 100 nm to about 600 nm.

149. The method of claim 148, wherein the exposing is performed using a mirror, a mirror array, a lens, a moving stage, a mask, or a scanning laser.

150. The method of claim 144, wherein the method further comprises hybridizing a cleavage probe to the cleavage domain of the capture probe in the area of the array not covered by the biological sample.

151. The method of claim 150, wherein the method further comprises contacting the biological sample with an enzyme that cleaves the cleavage domain hybridized to the cleavage probe.

152. The method of claim 150, wherein the hybridization of the cleavage probe to the cleavage domain results in formation of a cleavage site comprising DNA, RNA, or a combination thereof

153. The method of claim 152, wherein the hybridization of the cleavage probe to the cleavage domain results in formation of a restriction endonuclease cleavage site.

154. The method of claim 152, wherein the hybridization of the cleavage probe to the cleavage domain results in a CRISPR/Cas cleavage site.

155. The method of claim 151, wherein the enzyme is a DNase, an RNase, a restriction endonuclease, or a CRISPR/Cas cleavage enzyme.

156. The method of claim 150, further comprising removing unhybridized cleavage probes from the array.

157. The method of claim 144, wherein releasing the cleaved capture probes further comprises removing the cleaved capture probes from the array.

158. The method of claim 157, wherein the removing comprises washing the array.

159. The method of claim 144, wherein the analytes comprise DNA.

160. The method of claim 144, wherein the analytes comprise RNA, and optionally, comprise mRNA.

161. The method of claim 144, wherein the biological sample is a tissue sample or a tissue section.

162. The method of claim 161, wherein the tissue section is a fresh-frozen tissue section or a fixed tissue section.

163. The method of claim 144, wherein the array comprises one or more features.
